(19) United States
(12) Patent Application Publication
(10) Pub. No.: US 2026/0316009 A1
(43) Pub. Date: Oct. 15, 2026

(54) MICROFILTRATION DEVICE, METHOD OF PREPARING THE SAME, METHOD OF SEPARATING BIOLOGICAL MATERIAL AND A KIT

(71) Applicant: Massachusetts Institute of Technology, Cambridge, MA (US)

(72) Inventors: Jongyoon Han, Cambridge, MA (US); Yaoping Liu, Singapore (SG); Joshua Jebaraj Raymond, Singapore (SG)

(73) Assignee: Massachusetts Institute of Technology, Cambridge, MA (US)

(21) Appl. No.: 19/116,521

(22) PCT Filed: Oct. 2, 2023
(86) PCT No.: PCT/US2023/034274

§ 371 Date: Mar. 28, 2025

Related U.S. Application Data
(60) Provisional application No. 63/377,775, filed on Sep. 30, 2022.

(51) Int. Cl. B01D 61/14; B01D 65/02; B01D 67/00; B01D 69/02; B01D 71/08; C12N 1/16; C12N 1/20; C12N 7/00

(52) U.S. Cl. CPC B01D 61/147; B01D 65/02; B01D 67/0006; B01D 67/0088; B01D 69/02; B01D 71/08; C12N 1/16; C12N 1/20; C12N 7/00; B01D 2323/21817; B01D 2323/30; B01D 2325/02834; B01D 2325/04; B01D 2325/16; C12N 2710/16651

(57) ABSTRACT

Disclosed herein is a microfiltration device comprising a microfilter coated with a positively-charged degradable layer and a method of preparing the microfiltration device as described herein. Disclosed herein is also a method of separating a biological material using the microfiltration device as described herein and a kit comprising the microfiltration device as described herein. The microfiltration device and method of separating a biological material described herein demonstrates good compatibility with various downstream analysis techniques.

### REFERENCES TO RELATED APPLICATION

This application claims priority to U.S. application 63/377,775 filed with the United States Patent and Trademark Office on 30 Sep. 2022, the contents of which are hereby incorporated by reference.

### FIELD OF THE INVENTION

The present invention relates generally to a microfiltration device for separation of a biological material and a method of preparing the microfiltration device as described herein. The present invention also relates to a method of separation of a biological material using the microfiltration device as described herein and a kit for separation of the biological material comprising the microfiltration device as described herein.

### BACKGROUND

Detection and analysis of biological materials, especially microorganisms (bacteria, fungi, viruses) from various samples including biofluids, wastewater, foodborne specimens, and biomanufacturing products are important for both clinical diagnosis and surveillance of microorganisms. However, the low abundance of microorganisms against a huge number of backgrounds in a large-volume sample critically limits the detection performance of even the most sensitive techniques such as digital polymerase chain reaction (dPCR).

Commonly used techniques for microorganism separation include size-based separation techniques such as centrifugation and hollow-fibre filtration, however, these size-based separation techniques suffer from major drawbacks. Centrifugation is labour-extensive and suffers from serious loss of low-abundance microorganisms while hollow-fibre filtration is affected by serious clogging of complex contents in the crude samples and substantial loss of low-abundance microorganisms in the recovery step because of non-specific adhesion of the microorganisms.

Besides the above size-based separation techniques, there also have been electrostatic charge-based methods to separate and recover microorganism from liquid/water samples such as negative electrostatic filtration and positive electrostatic filtration. To avoid the need for pre-conditioning step associated with negative electrostatic filtration, positive electrostatic filtration was typically used as an alternative, however, this technique mainly suffered limitations of traditional filtration membranes, such as biofouling and clogging. Furthermore, these traditional electro-positive filters still suffer from the bottleneck of concentration because the elution solution volume is large (~10 mL), and therefore requires a second concentration for enriching the analytes (microorganism) of interest.

Besides the above shortcomings, the release and recovery of captured microorganisms from the above-mentioned filters is very difficult and of low efficiency due to the strong electrostatic attraction between the microorganisms and the filter that is hard to break or overcome. Additionally, the sponge-like structures of these filters limit the efficient release of captured microorganisms, thus a bottleneck is established between separation and analysis time.

There is thus a need to provide for a microfiltration device that is able to enrich microorganism recovery to improve downstream detection, even when the microorganisms are low in abundance. There is also a need to provide for a method of separation that is able to interface with downstream detection methods such that minimal time is spent between separation and analysis.

### SUMMARY

In one aspect, the present disclosure refers to a microfiltration device for separation of a biological material from a sample, comprising a microfilter coated with a positively-charged degradable layer.

Advantageously, the microfiltration device described herein may be used for the separation and recovery of various biological materials. In particular, the microfiltration device described herein was found to be effective for the separation of various microorganisms, including bacteria, fungi and viruses. The effective recovery of the various microorganisms may be attributed to the positively-charged degradable layer coated on the microfilter, which allows for separation to be achieved by electrostatic interactions in addition to size exclusion. Accordingly, the microfiltration device and method described herein provides a versatile sample preparation method to capture and concentrate biological materials for downstream processing.

In another aspect, the present disclosure refers to a method of preparing a microfiltration device for separation of a biological material from a sample, the method comprising the step of coating a positively-charged degradable layer onto the surface of a microfilter.

In another aspect, the present disclosure refers to a method of separating a biological material using the microfiltration device as described herein, the method comprising the step of passing a sample comprising the biological material through the microfiltration device as described herein, whereby the biological material is captured on the positively-charged degradable layer on the microfilter, thereby separating the biological material from the sample.

Advantageously, the method of separating a biological material with the microfiltration device described herein achieves high capture efficiency, even for samples comprising low concentrations of biological material. In one example, capture efficiencies of about 85% may be achieved even for samples comprising 1 Colony Forming Unit (CFU)/mL bacteria. The high capture efficiencies achieved with the microfiltration device may be attributed to the positively-charged degradable material coated on the microfilter. Specifically, the negatively charged biological material may form strong electrostatic interactions with the positively-charged degradable layer coated on the microfilter, allowing effective separation of the negatively charged biological material from the sample.

Advantageously, the method of biological material separation as described herein affords high capture efficiency (>90%) and high enrichment factor (higher than 150× and may increase to 8333×) due to the smaller volume of solution (~60 μL used herein) used to release/recover the captured biological materials from the positively-charged degradable layer. In doing so, it would be understood that the biological material will be concentrated. Accordingly, where the biological material is recovered using a volume which is smaller than the input volume, the method of separating a biological material may also be interpreted to be a method of concentrating a biological material via electrostatic microfiltration (i.e. EM concentration).

Further advantageously, the method of biological material separation using the microfiltration device as described herein affords lower limits of detection (~100× lower) when using digital loop-mediated isothermal amplification (dLAMP). The samples (>10 mL) of 1~1,000 CFU/mL are not detectable based on the current centrifugation-based protocols but can become detectable via dPCR based on the invented high-throughput (≥1 mL per minute) electrostatic microfiltration enabled concentration.

Still further advantageously, the method of biological material separation as described herein allows it to be compatible with other downstream methods, such as polymerase chain reaction (PCR)-based methods, sequencing, and MALDI-TOF mass spectrometry etc., because of the high enrichment factor which enables good compatibility to interface with the downstream analysis after separation, and reduces the processing time as there is no need for further enrichment steps.

In another aspect, the present disclosure refers to a kit comprising the microfiltration device as described herein, a sampling tube, a drainage tube and a housing container.

The present disclosure also refers to a microfluidic capture device comprising a positively-charged degradable layer.

The present disclosure also refers to a microfilter coated with a positively-charged degradable layer.

The present disclosure also refers to a kit-of-parts comprising a microfilter having a positively-charged degradable layer thereon, a sampling zone and a collection zone.

### BRIEF DESCRIPTION OF DRAWINGS

The invention will be better understood when considered in conjunction with the non-limiting examples and the accompanying drawings, in which:

FIG. 1 shows a schematic diagram of the working principle of a microfiltration device comprising a microfilter coated with a positively-charged degradable layer and its use in a method of for separating biological material from a sample. Row a) of FIG. 1 shows the preparation of a positively-charged degradable layer 117 from a polymer substrate solution 101 and a cross-linking agent 103 and the effect of contacting the positively-charged degradable layer 117 with a degradation agent 109. Row b) of FIG. 1 shows the coating of a microfilter 115 with the positively-charged degradable layer 117, and the removal of the positively-charged degradable layer 117 using a degradation agent 109, while row c) of FIG. 1 illustrates a method of separating biological material using the microfiltration device 106 and the subsequent steps of removing the concentrated or separated biological material 125 from the microfilter 115.

FIG. 2a shows a scanning electron microscope (SEM) image of the micropores of the microfilter before calcium alginate gel coating, FIG. 2b shows a SEM image of the micropores with calcium alginate gel coating and FIG. 2c shows a SEM image of the micropores after degrading the coated calcium alginate gel with EDTA (or alginate lyase) incubation. All scale bars are 5 μm.

FIG. 3a shows a schematic diagram of a method of separating a biological material using the microfiltration device as described herein with additional processing steps for the detection of the captured biological material such as bacteria, according to Example 2. The method may comprise the steps of a) introducing a sample comprising biological material to the microfiltration device; b) passing the sample through the microfiltration device driven by gravity, optionally accelerated by an applied force (e.g. centrifugation, pressure or vacuum); c) removing the microfilter from the microfiltration device and contacting the microfilter comprising the captured bacteria with a lysis buffer; d) inducing lysis of the captured bacteria through a cycle of vortexing and heating; e) separating the supernatant from the waste pellet and f) detecting the presence of the bacteria using a dLAMP procedure. FIG. 3b is a photo of a microfiltration device as described herein. The microfiltration device may comprise a sampling container 317, a securing means 319 to attach the support 321 to the sampling container 317, and a drainage tube 323. FIG. 3c is a top-down view of a microfilter 325 on the microfilter support 321 which may comprise an opening 327, over which microfilter 325 may be placed. FIG. 3d is a side view of the microfiltration device as described herein. FIG. 3e is a photo of the method of separating biological as described herein, where a sample may be introduced into the sampling container 317, and passed through the microfilter 325 secured on the support 321. Gravity-driven filtration of a 10 mL sample may be achieved within just 1.5 min, using the microfiltration device described herein.

FIG. 4a shows a fluorescence distribution image of a dLAMP detection signal for nucleic acid obtained from electrostatic microfiltered bacteria according to Example 2 and FIG. 4b shows a fluorescence distribution image of the dLAMP detection signal for nucleic acid obtained from conventional centrifugation technique (10,000×g for 10 minutes).

FIG. 5a shows a scatter plot of partitions in the dLAMP detection for nucleic acid obtained from electrostatic microfiltered bacteria according to Example 2 and FIG. 5b shows a scatter plot of partitions in the dLAMP detection for nucleic acid obtained from conventional centrifugation technique (10,000×g for 10 minutes). The x axis is the partition number and the y axis is the fluorescence intensity in relative fluorescence units.

FIG. 6 shows a SEM image of electrostatically captured S. aureus (Sa.6538, ATCC) on the alginate gel layer of the microfilter (scale bar of 5 μm) according to Example 2

FIG. 7a shows a SEM image of bacteria (S. aureus, K. pneumoniae, P. aeruginosa) captured on the alginate layer, with a scale bar of 10 μm, according to example 4. FIG. 7b shows a bar chart of the bacterial DNA of S. aureus (□Sa) K. pneumoniae (□Kp) and P. aeruginosa (□Pa), as detected by dLAMP in a sample comprising a mixture of S. aureus, K. pneumoniae and P. aeruginosa at various concentrations, according to example 4. FIG. 7c shows a SEM image of fungi (C. albicans) captured on the alginate layer, with a scale bar of 10 μm; while FIG. 7d shows a SEM image of Herpes simplex virus (HSV) particles captured on the alginate layer according to Example 7, with a scale bar of 1 μm. SEM images of the captured HSV of 10× higher magnification (scale bar of 100 nm) is inlaid in FIG. 7d.

FIG. 8a shows the SYTO9 (live and dead) signal of the bacteria (K. pneumoniae) in a control sample which was not subjected to filtration or release, while FIG. 8b shows the PI (dead) signal of the bacteria from the same control sample. FIG. 8c shows the SYTO9 (live and dead) signal of the bacteria (K. pneumoniae) released from the alginate gel after filtration of a sample through the microfiltration device described herein, while FIG. 8d shoes the PI (dead) signal of the bacteria captured and released from the alginate gel after filtration of the same sample according to Example 3.

FIG. 9a is a photograph of an LB-agar plate with cultured colonies of K. pneumoniae recovered from the microfiltration device. FIG. 9b is a photograph of an LB-agar plate with cultured colonies of P. aeruginosa, recovered from the alginate gel of the microfiltration device described herein. FIG. 9c is a photograph of an LB-agar plate with cultured colonies of S. aureus according to Example 3.

FIG. 10a is a bar graph indicating recovery efficiencies from samples having a low abundance of S. aureus bacteria using the method of separating biological material described herein, FIG. 10b is a bar graph of the recovery efficiencies of K. pneumoniae from samples comprising a low abundance of the bacteria using the method of separating biological material described herein, while FIG. 10c is a bar graph of the recovery efficiencies from Luria Borth (LB) samples comprising a low abundance of P. aeruginosa bacteria, using the electrostatic microfiltration method described herein, according to Example 2. The recovery efficiencies of the electrostatic microfiltration method described herein, indicated with solid coloured bars (□), is compared to the recovery efficiencies of conventional centrifugation methods, indicated with bars with diagonal lines (□). The x-axis of the graph is the concentration of the microorganism in the sample, while the y-axis of the graph is the recovery efficiency of the microorganism from the sample. FIG. 10d is a scatter plot of the dLAMP signals of the bacterial DNA of recovered S. aureus from samples comprising a low abundance of the bacteria, using the separation method described herein, FIG. 10e is a box chart of the median and range within 1.5 Interquartile Range (IQR) of the dLAMP signals of the bacterial DNA of recovered S. aureus from samples comprising a low abundance of the bacteria, using the separation method described herein as compared to traditional centrifugation methods and raw sampling. FIG. 10f is a scatter plot of the dLAMP signals of bacterial DNA of recovered K. pneumoniae from samples comprising a low abundance of the bacteria using the method of separating biological material described herein as compared to traditional centrifugation methods and raw sampling.;

FIG. 10g is a box chart of the median and range within 1.5 IQR of the dLAMP signals of bacterial DNA of recovered K. pneumoniae from samples comprising a low abundance of the bacteria using the method of separating biological material described herein as compared to traditional centrifugation methods and raw sampling. FIG. 10h is a scatter plot of the dLAMP signals of bacterial DNA of recovered P. aeruginosa from samples comprising a low abundance of the bacteria using the method of separating biological material described herein, as compared to traditional centrifugation methods and raw sampling. FIG. 10i is a box chart of the median and range within 1.5 IQR of the dLAMP signals of bacterial DNA of P. aeruginosa recovered from samples comprising a low abundance of the bacteria using the method of separating biological material described herein as compared to traditional centrifugation methods and raw sampling. This is obtained according to Example 2. The x-axis is the abundance of the bacteria in the sample, while the y-axis is the dLAMP signal readout. Samples with 0 CFU/mL or NTC bacteria indicate that no microorganism was contained in the sample. In each of FIG. 10d, FIG. 10f and FIG. 10h, the dLAMP readings of bacterial DNA detected in each replicate using the method of separating biological material described herein is indicated with a clear square marker (□); the dLAMP signals of bacteria DNA detected in each replicate using conventional centrifugation methods is indicated with a clear, triangle marker (Δ), while the dLAMP readings of bacterial DNA detected in each raw sample replicate, which is not subjected to filtration, is indicated with a clear, star-shaped marker (☆). In each of FIG. 10e, FIG. 10g and FIG. 10i, the average dLAMP signal for bacterial DNA detected in samples of bacteria recovered using the method of separating biological material described herein is indicated with a square solid marker (▪); the average dLAMP signal of bacterial DNA detected in samples of bacteria recovered from the samples using conventional centrifugation methods is indicated with a solid triangle marker (▴), the average dLAMP signals of bacterial DNA in the raw sample comprising the bacteria, which is not subjected to any filtration or separation method, is indicated with a solid, star-shaped marker (★). In each box chart of FIG. 10e, FIG. 10g and FIG. 10i, boxes drawn with solid outlines but no pattern fill (□) are indicative of the dPCR or dLAMP data for samples of bacteria recovered using the method of separating biological material described herein; boxes drawn with solid outlines and horizontal lines fill (□) represent the dPCR or dLAMP data for samples of bacteria recovered using traditional centrifugation methods; while boxes drawing with solid outlines with diagonal brick fill (□) represent the dPCR or dLAMP data for raw samples of bacteria. The height of the box about each marker indicates the range of the 1.5 IQR of the dLAMP signals obtained for the replicates, and the line within the box marks the median value of the dLAMP signals. Bars which extend from the box indicate the range of the remaining signals. Data sets marked with brackets and a * indicate that the significance test P value is smaller than 0.05, and the limit of detection may be determined based on the significance test (P<0.05) relative to the signals of NTC group.

FIG. 11a is a scatter plot of the dLAMP signals of detected bacterial DNA of recovered S. aureus using the method of separating biological material described herein, FIG. 11b is a scatter plot of the dLAMP signals of bacterial DNA of recovered K. pneumoniae using the method of separating biological material described herein, while FIG. 11c is a scatter plot of the detected bacterial DNA of recovered P. aeruginosa using the method of separating biological material described herein. The x-axis is the abundance of the bacteria in the sample, while the y-axis is the dLAMP signal readout. The dLAMP signals of the bacterial DNA of bacteria recovered from samples of the bacteria in LB only is indicated with a clear, square marker (□), while the dLAMP signals of the bacterial DNA of bacteria recovered from samples comprising the bacteria in LB containing serum are indicated with a clear round marker (⊗). This was obtained according to Example 5.

FIG. 12a is a scatter plot of the bacterial DNA of S. aureus detected by dLAMP after recovery of the bacteria from a 100 mL and 500 mL sample using the method of separating biological material described herein, FIG. 12b is a scatter plot of the bacterial DNA of K. pneumoniae detected by dLAMP after recovery of the bacteria from a 100 mL and 500 mL sample of water, using the method of separating biological material described herein, while FIG. 12c is a scatter plot of the bacterial DNA of P. aeruginosa recovered from a 100 mL and 500 mL water sample using the method of separating biological material described herein. The x-axis is the abundance of the bacteria in the water sample, while the y-axis is the dLAMP signal readout. The dLAMP signal of the bacterial DNA detected after recovery of the bacteria from a 100 mL water sample for each replicate is indicated with a clear, round marker (⊗). The average dLAMP signal of the bacterial DNA detected after recovery of the bacteria from a 500 mL water sample is indicated with a clear, square marker (□). This was obtained according to Example 6.

FIG. 13a is a scatter plot of the fungal DNA detected by dLAMP after recovery of C. albicans from a 10 mL sample of Brain Heart Infusion (BHI) broth using the method of separating biological material described herein. FIG. 13b is a scatter plot of the dPCR signals of viral DNA upon recovery of herpes simplex virus (HSV) from cell spent media (DMEM) samples containing HSV, using the method of separating biological material described herein. The clear, square markers (□) are the dLAMP or dPCR signals of each replicate. This was obtained according to Example 7.

### DEFINITIONS

The term “biological material” as used herein refers to, but is not limited to, microbes, microorganisms (or cells thereof which may include bacteria, fungi and viruses), pathogens, nucleic acids (such as cell free nucleic acids), proteins and exosomes.

The term “microorganism” refers to organisms of microscopic sizes including, but not limited to, bacteria, fungi, viruses, protozoa, algae and parasites, which may or may not be capable of causing disease.

The term “pathogen” as used herein refers to a microorganism such as bacteria, fungi, viruses or any other microorganism that is capable of causing disease.

The term “Parylene” refers to the common name of a polymer whose backbone consists of para-benzenediyl rings —C₆H₄— connected by 1,2-ethanediyl bridges —CH₂—CH₂—. It can be obtained by polymerization of para-xylylene H₂C═C₆H₄═CH.

The term “enrichment factor” refers to the input/starting sample volume divided by the volume used to recover or re-suspend the (e.g. microorganisms) biological material (e.g. microorganisms) retained on or retained by the microfilter after passing the sample through the microfiltration device.

The term “porosity” as used herein refers to the fraction of the area of the pore openings over the total area of the microfilter.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### DETAILED DESCRIPTION OF OPTIONAL EMBODIMENTS

Exemplary, non-limiting embodiments of a microfiltration device for separating biological material from a sample, wherein the microfiltration device comprises a microfilter coated with a positively-charged degradable layer will now be disclosed.

The microfiltration device may comprise a microfilter. The microfilter may comprise pores, preferably an array of micropores, adapted for size-exclusive filtration.

The microfilter may be made from a biocompatible material that does not react adversely with biological materials such as biomolecules and microorganisms, including pathogens. The biocompatible material may be an FDA-approved (U.S. Food and Drug Association) biocompatible material. The biological material does not degrade or become inactivated upon contact with the material. Where the biological material is a microorganism, such as a pathogen, the microorganism or pathogen remains viable upon contact with the material, which can then be used for downstream studies which require the microorganism or pathogen to be viable.

The microfiltration device described herein was surprisingly found to be effective for the recovery of various microorganisms, including bacteria, fungi and viruses. This may be attributed to the working principle of the microfilter, which relies upon electrostatic interactions between the negative charge on the cell surfaces of the microorganisms and the positively-charged degradable layer for isolation of the microorganisms.

The microfilter may be made from an inert, biocompatible material such as an inert, biocompatible polymer. The polymer may be an unsubstituted, hydrocarbon polymer. The microfilter may be made from a biocompatible polymer selected from Parylene C, Parylene N, Parylene D, Parylene AF-4 and combinations thereof. The biocompatible material may be Parylene C.

The material of the microfilter may also be chemically inert and therefore, compatible with various downstream chemical treatments or chemical extractions.

The microfilter may have a thickness in the range of about 4 micrometres (μm) to about 10 micrometres, about 6 micrometres to about 10 micrometres, about 8 micrometres to about 10 micrometres, about 4 micrometres to about 8 micrometres, or about 4 micrometres to about 6 micrometres. The thickness of the microfilter may be about 4 micrometres, 4.2 micrometres, 4.4 micrometres, 4.6 micrometres, 4.8 micrometres, 4.9 micrometres, or 5 micrometres.

The microfilter may comprise pores, or an array of pores adapted for separation of biological materials from a suspension. The pores may have an average diameter in the range of about 1 micrometre to about 10 micrometres, about 2 micrometres to about 10 micrometres, about 4 micrometres to about 10 micrometres, about 6 micrometres to about 10 micrometres, about 8 micrometres to about 10 micrometres, about 1 micrometre to about 8 micrometres, about 1 micrometre to about 6 micrometres, about 1 micrometre to about 4 micrometres, or about 1 micrometre to about 2 micrometres. For example, the average diameter of the micropores may be about 1 micrometre, 1.2 micrometres, 1.4 micrometres, 1.5 micrometres, 1.6 micrometres, 1.8 micrometres or 2 micrometres.

Upon coating of the microfilter with the positively charged degradable layer, the average pore size of the coated microfilter may decrease. The pores of the coated microfilter have an average diameter of about 1 micrometre to about 10 micrometres, about 2 micrometres to about 10 micrometres, about 4 micrometres to about 10 micrometres, about 6 micrometres to about 10 micrometres, about 8 micrometres to about 10 micrometres, about 1 micrometre to about 8 micrometres, about 1 micrometre to about 6 micrometres, about 1 micrometre to about 4 micrometres, or about 1 micrometre to about 2 micrometres. For example, the average diameter of the pores of the microfilter coated with the positively charged degradable layer may be about 1.2 micrometres.

The average diameter of the pores of the microfilter may be about 1.5 micrometres. The pore-to-pore space of the microfilter may be about 1 micrometre to about 10 micrometres, 1 micrometre to about 9 micrometres, 1 micrometre to about 8 micrometres, 1 micrometre to about 7 micrometres, 1 micrometre to about 6 micrometres, 1 micrometre to about 5 micrometres, 1 micrometre to about 4 micrometres, 1 micrometre to about 3 micrometres, 2 micrometres to about 3 micrometres. For example, the pore-to-pore space may be about 1.5 micrometres, 1.8 micrometres, 2 micrometres, 2.5 micrometres or 3 micrometres. The pore-to-pore space of the microfilter may be 2.5 micrometres.

The microfilter may have a pitch of about 2 micrometres to about 6 micrometres, about 4 micrometres to about 6 micrometres, or about 2 micrometres to about 4 micrometres. The pitch of the microfilter may preferably be about 3 micrometres to about 5 micrometres, such as, 3 micrometres, 3.2 micrometres, 3.5 micrometres, 3.8 micrometres, 4 micrometres, 4.2 micrometres, 4.5 micrometres, 4.8 micrometres, or 5 micrometres. The pitch of the microfilter may be about 4 micrometres. The microfilter may be termed as a micropore array.

The pitch (pore diameter, pore-to-pore space) of the microfilter may be optimised to afford higher throughput microfiltration. As used herein, the term “pitch” refers to distance between repeated elements in a structure with translational symmetry. With regard to the microfilter above, which may be described as a micropore array, the pitch may refer to the distance between the centres of adjacent pores or the distance from one edge of a pore to the equivalent edge of the next pore. This may therefore be considered as being mathematically equivalent to the sum of the average pore diameter and the space between the pores of microfilter. The spaces between the pores of the microfilter may be optimized to adjust the porosity of the microfilter, in order to filter a larger sample volume. In particular, the smaller the space between the pores of the microfilter, the higher the porosity of the micropore array and thus, a higher throughput of microfiltration. This may enable a larger sample volume to be processed within a certain time period.

The porosity of the microfilter may be more than 10%, more than about 14%, more than 20%, more than 25%, more than about 30%, more than about 40%, more than about 50%, more than about 60%, more than about 70%, more than about 80%. The porosity of the microfilter may be in the range of about 10% to about 90.9%, such as about 12%, 14%, 20%, 25%, 30%, 35%, 40%, 45%, 50%, 55%, 60%, 65%, 70%, 75%, 80%, 85% or 90%. The porosity of the microfilter may be about 14%.

The porosity of the microfilter may change upon coating with the positively charged degradable layer. The porosity of the microfilter coated with the positively charged degradable layer may be lower than the porosity of the microfilter prior to the coating. For example, the porosity of the microfilter coated with the positively charged degradable layer may be more than about 5%, more than about 10%, more than about 20%, more than about 30%, more than about 40%, or more than about 50%. The porosity of the coated microfilter may be in the range of about 5% to about 90.9%, such as about 6%, 8%, 10%, 25%, 30%, 35%, 40%, 45%, 50%, 55%, 60%, 65%, 70%, 75%, 80%, 85% or 90%. The porosity of the microfilter coated with the positively charged degradable layer may be 9% or more, or about 9%.

The porous microfilter may allow the sample to pass through the microfilter rapidly and with minimal clogging. As an example, it can take less than 30 minutes to process a sample volume of more than 100 mL using a microfilter coated with a positively-charged degradable layer having ~1.2 μm pore size and ~2.8 μm pore-to-pore space (i.e., 4 μm pitch), and a porosity of about 9%.

Advantageously, the microfilter of the present disclosure has a high filtration rate due to its thickness (5 μm) and porosity (≥9%). In particular, the porosity of the microfiltration device described herein allows for filtration of large volumes of sample. For example, the microfiltration device may be used to filter about 500 mL of sample. The ability to filter large volumes is particularly advantageous for the recovery of biological material such as microorganisms provided in low concentrations. In particular, the microfilter allows for effective recovery of microorganisms from samples of large volume which may comprise low concentrations of such microorganisms. In one example, the microfiltration device described herein enables a limit of detection (LOD) of as low as 10 CFU to be achieved for filtration of a sample of 500 mL.

Further advantageously, the Parylene C material of the microfilter ensures that the microfilter remains chemically inert, thus making it compatible with various downstream chemical treatments, biological studies or nucleic acid extraction. Further, Parylene C is an FDA-approved biocompatible material, thus ensuring the viability of the separated biological materials.

The microfilter may not be limited to a specific size or shape and may be modified or adapted based on the dimensions of the microfiltration device, sampling zone, the collection zone or the housing. The microfilter may be provided in a round shape, or any quadrilateral shape such as a square or rectangle, Where the microfiltration device is housed in a centrifuge tube or microcentrifuge tube, the size of the microfilter may be adjusted based on the inner diameter of the housing. For example, where the microfiltration device is housed in a Falcon tube, the dimensions of the microfilter may be adjusted to about 17 mm by 17 mm. The microfiltration device may be provided in the form of a gravity-driven filtration device, wherein the microfilter may be provided within an insert which may be contained within the housing.

The positively-charged degradable layer may comprise a polymer substrate. The polymer substrate may be a gel, including biocompatible hydrogels. Such gels may include gelatin, agar, iota carrageenan gel, alginate gel and naturally occurring gums such as Gellan gum, Xanthan gum, Guar gum, Gum arabic and Acacia gum. The polymer substrate may be an alginate gel or an iota carrageenan gel. In an example, the polymer substrate may be alginate gel. Therefore, the positively-charged degradable layer may be termed as a positively-charged alginate gel layer.

The polymer substrate may be modified with a cation, such as a monovalent or divalent cation to form the positively-charged layer. The cation may form cross-links in the polymer substrate to impart a net positive charge, thereby yielding the positively-charged degradable layer.

The positively-charged degradable layer may comprise a divalent cation. The divalent cation may be selected from the group consisting of calcium (II) (Ca²⁺), strontium (II) (Sr²⁺), barium (II) (Ba²⁺), iron (II) (Fe²⁺), copper (II) (Cu²⁺), zinc (II) (Zn²⁺) and mixtures thereof. The divalent cation may be calcium (II) (Ca²⁺). Alternatively, or additionally, the positively-charged degradable layer may comprise a monovalent cation, which may include potassium (K⁺), sodium (Na⁺), lithium (Li⁺), copper (I) (Cu⁺) or mixtures thereof. The positively-charged degradable layer may comprise a mixture of the divalent cation and the monovalent cation, each being independently selected.

Advantageously, the presence of a divalent cation imparts a strong net positive charge on the positively-charged degradable layer. This may contribute to stronger attraction of the biological material to the positively-charged degradable layer, thereby improving the separation of the biological material from a sample, even for samples with low concentrations of the biological material.

Where the polymer substrate is an alginate, the introduction of calcium ions to the alginate solution facilitates the formation of a calcium-alginate gel due to the formation of crosslinks with the calcium ions. The presence of the calcium cations in the gel also imparts a net positive charge on the alginate.

The positively-charged degradable layer can be regarded as being coated on the microfilter and can be referred to as a positively-charged degradable coating. The positively-charged degradable layer may be in the form of a gel or a gel layer. The positively-charged degradable layer may be a controllably degradable hydrogel (as will be explained in further detail below).

The positively-charged degradable layer coated on the microfilter may have a thickness in the range of about 0.2 micrometres to about 2 micrometres, about 0.5 micrometres to about 2 micrometres, or about 1 micrometre to about 2 micrometres. For example, the positively-charged degradable layer may have a thickness of about 0.5 micrometre, 0.75 micrometre, 1 micrometre, 1.2 micrometres, 1.4 micrometres, 1.5 micrometres, 1.6 micrometres, 1.8 micrometres or 2 micrometres. The thickness of the positively-charged degradable layer may be 1.25 micrometres.

The positive charge on the degradable layer may be removed by reacting the positively-charged degradable layer with a suitable degradation agent that reacts with the cation on the degradable layer to form a complex and at the same time, dissolves the degradable layer to form the polymer substrate or a solution of the polymer substrate. Therefore, the term “degradable” refers to the removal of the cation from the degradable layer and the dissolving of the degradable layer into a solution of the polymer substrate due to reaction with the degradation agent. The degradation can be controllable as this depends on when the degradation agent is added. The degradation agent may be alginate lyase, ethylenediaminetetraacetic acid (EDTA), egtazic acid (EGTA), sodium citrate, nitrilotriacetic acid, or n-hydroxyethylethylenediaminetriacetic acid (hEDTA). The degradation agent may additionally or alternatively be an organic acid such as oxalic acid, malic acid, rubeanic acid or citric acid. The degradation agent may be EDTA or alginate lyase. Where alginate is used, the positively-charged alginate gel layer may be termed as a degradable positively-charged alginate gel layer.

Due to the positive charge on the positively-charged degradable layer, biological material that is negatively charged or carries a negative charge (for brevity, this will be termed as “negatively-charged biological material” herein) will be attracted to the positively-charged degradable layer by electrostatic attraction or interactions and thereby be captured on the microfilter. The biological materials that are not negatively-charged (such as biological materials that are positively-charged or are not charged) may be captured on the microfilter by size exclusion or simply pass through the microfilter. The separation by electrostatic interactions described herein was found to be surprisingly effective for the capture of various microorganisms, including bacteria, fungi and viruses.

Advantageously, the separation of the biological material by electrostatic interactions allows for the recovery of whole microorganisms, even in the presence of other biomaterials. In particular, the microfiltration device described herein was found to be effective for the recovery of microorganisms in samples containing serum, proteins and other nutrients typically found in biological samples. In one example, the recovery of microorganisms from samples comprising a high concentration of protein was found to be comparable to the recovery of microorganisms from cell culture broth. This is believed to be due to the electrostatic interaction between such microorganisms (having negative-charged cell surfaces) with the positively-charged degradable layer which facilitates separation by electrostatic interactions.

The microfiltration device may be a gravity-driven microfiltration device. The microfiltration device may comprise the microfilter with the positively-charged degradable layer thereon, a sampling zone where the sample may be introduced and passed through the microfilter and a collection zone on the opposite side of the microfilter for collecting the filtrate or filtered sample, whereby the filtered sample has a decreased amount or number of biological material as compared to the (initial) sample. For example, the sampling zone may be the area above the surface of the positively-charged degradable layer, while the collection zone may be located below the microfilter. The sampling zone may be located within a sampling container such as a sampling bowl, while the collection zone may be contained within a collection container, such as a collection tube. The microfiltration device may also comprise a support comprising an opening, over which the microfilter may be placed or attached. The support may comprise a drainage tube which may direct the flow of the filtrate to the collection container. The support may be integrally formed with the sampling container, or may be provided as a separate component, which is detachable from the sampling container. A securing means may also be provided on the support to attach the support to the sampling container. The securing means may also be used to secure the microfilter over the opening of the support. This may prevent movement of the microfilter during the method of separating the biological material.

The microfiltration device may be adapted based on the sample to be separated. Accordingly, the dimensions of the microfiltration device, including the microfilter, sampling container, collection container, support, opening on the support, drainage tube and securing means may be provided in any shape or size. For example, the width of the opening provided on the support may be adapted to facilitate high throughput and flow of the filtrate into the collection tube. The width of the opening may be smaller than the width of the microfilter. For example, where a microfilter having a width of 17 mm is provided, the opening may have a width of 13 mm, and the diameter of the drainage tube may be 13 mm.

After passing the sample through the microfiltration device, the microfilter with the positively-charged degradable layer may be removable from the microfiltration device and then treated with the degradation agent to release the negatively-charged biological material from the microfilter. Alternatively, the degradation agent may be added to the sampling zone of the microfiltration device and the negatively-charged biological material released from the degradation of the positively-charged layer collected from the collection zone of the microfiltration device. The negatively-charged biological material can then be processed downstream as needed.

Exemplary, non-limiting embodiments of a method of preparing a microfiltration device for separating biological material from a sample will now be disclosed.

The method may comprise the step of coating a positively-charged degradable layer onto the surface of a microfilter. The positively-charged degradable layer may be a positively-charged alginate gel layer.

The method may further comprise the steps of preparing the positively-charged degradable layer by mixing a crosslinking agent with a polymer substrate solution such as an alginate solution. The step of preparing a mixture comprising a crosslinking agent and a polymer substrate solution may be performed prior to the coating step.

The crosslinking agent may be a metal salt, wherein the metal may be selected from the group consisting of calcium (Ca), strontium (Sr), barium (Ba), iron (Fe), copper (Cu), zinc (Zn), potassium (K), sodium (Na) and lithium (Li⁺). The metal salt may be a metal salt which is soluble in water. For example, the metal salt may be a metal halide salt such as a metal chloride salt, a metal bromide salt or a metal iodide salt. The metal salt may also be a metal nitrate salt, metal sulfate salt, metal phosphate salt, a metal hydrogen carbonate salt or a metal carbonate salt which is soluble in water. The crosslinking agent may be CaCl₂.

The alginate solution may comprise an alginate precursor. The alginate precursor may be selected from the group consisting of alginic acid, sodium alginate, potassium alginate, ammonium alginate and propylene glycol alginate. The alginate solution may be sodium alginate solution.

The coating step may be carried out by spin coating the mixture comprising the crosslinking agent and the polymer substrate solution onto the surface of the microfilter to thereby coat the surface of the microfilter with the positively-charged degradable layer. The positively-charged degradable layer may be coated onto the microfilter by spin coating at rotations per minute (rpm) of about 500 rpm to about 2 000 rpm, about 1 000 rpm to about 2 000 rpm, about 1 500 rpm to about 2 000 rpm, about 500 rpm to about 1 500 rpm, or about 500 rpm to about 1 000 rpm, for a duration of about 10 seconds to about 60 seconds, about 20 seconds to about 60 seconds, about 30 seconds to about 60 seconds, about 40 seconds to about 60 seconds, about 50 seconds to about 60 seconds, about 10 seconds to about 50 seconds, about 10 seconds to about 40 seconds, about 10 seconds to about 30 seconds, or about 10 seconds to about 20 seconds.

The spinning rate may be controlled to obtain a desired thickness of the positively-charged degradable layer. The spinning rate may preferably be 2 000 rpm.

The positively-charged degradable layer may comprise at least two sub-layers, each sub-layer being the same as or different from other. As an example, where there are three sub-layers of Ca²⁺-alginate, the positively-charged degradable layer can be regarded as a layer of Ca²⁺-alginate/Ca²⁺-alginate/Ca²⁺-alginate sub-layers where the total thickness of the entire layer may be in the range of 0.2 micrometre to 2 micrometres, 0.5 micrometres to about 2 micrometres, or about 1 micrometre to about 2 micrometres. Each sub-layer may be spun coated and left to rest before the next sub-layer is coated thereon.

Where the positively-charged degradable layer is the positively-charged alginate gel layer, the method may further comprise the step of setting the alginate gel on the microfilter by resting the gel. The coated microfilter may be rested for a duration of about 10 minutes to about 30 minutes, about 20 minutes to about 30 minutes, or about 10 minutes to 20 minutes in order to set the alginate gel layer.

Exemplary, non-limiting embodiments of a method of separating a biological material from a sample using the microfiltration device as described herein will now be disclosed.

The biological material to be separated may be negatively-charged. The biological material may be negatively charged biomolecules or whole microorganisms. The biological material which may be captured on the positively-charged degradable layer may be whole microorganisms, in particular, pathogenic microorganisms. The microorganisms may be bacteria, fungi or viruses. For example, the biological material may be pathogenic bacteria and fungi which carry a net negative charge on its cell wall or viruses comprising a capsid with a net negative charge. For example, the microfiltration device described herein may allow for capture of S. aureus, K. pneumoniae, P. aeruginosa, C. albicans, Herpes Simplex Virus (HSV).

The method of separating a biological material from a sample may comprise the step of passing the sample comprising the biological material through the microfiltration device as described herein, wherein the biological material is captured on the positively-charged degradable layer on the microfilter. The biological material may be negatively charged biological material. The passing step may comprise capturing or trapping the negatively charged biological material on the microfiltration device, specifically on the positively-charged degradable layer coated on the microfilter. For example, as the sample is passed through the microfiltration device, the negatively charged biological material may be captured by the positively-charged degradable layer, whereby the negatively-charged biological material may be attracted to the positively-charged degradable layer on the microfilter by electrostatic interactions, thereby separating the negatively-charged biological material from the sample.

The sample may comprise a liquid. The sample may have a volume in the range of about 100 millilitres to about 1 litre, about 200 millilitres to about 1 litre, about 400 millilitres to about 1 litre, about 800 millilitres to about 1 litre, about 100 millilitres to about 800 millilitres, about 100 millilitres to about 400 millilitres, or about 100 millilitres to about 200 millilitres.

The sample may be passed through the microfiltration device using gravity-driven filtration, ultrafiltration, vacuum filtration, centrifugation or any combination thereof.

Prior to passing the sample through the microfiltration device, the sample may undergo a pre-separation or pre-filtration step to remove larger-sized contents from the sample. The pre-separation step may include, but are not limited to, techniques based on multi-layer microfiltration, low-speed centrifugation, and any combination thereof.

The method may further comprise the step of isolating the microfilter comprising the negatively-charged biological material attracted thereon from the microfiltration device. The negatively-charged biological material may be isolated from the sample predominantly by size exclusion and, where applicable, also by charge attraction to the positively-charged layer. The negatively-charged biological material may preferably be isolated from the sample by charge attraction to the positively-charged alginate gel layer.

Where the biological material are microorganisms, there may be a further method of culturing the microorganisms (such as by inoculation/plating) comprising the steps of:
a) removing the separated microorganisms from the microfilter with a release solution; and
b) streaking the inoculation loop of the release solution with microorganisms in step a) onto a culture plate for functional tests.
   The “release solution” described herein may refer to a solution comprising the degradation agent and captured or separated microorganisms.

The culture plate of step b) may comprise an agar selected from the group comprising of Luria agar (LB agar) and Mueller-Hinton agar (MH agar) or any other agar that is suitable for culturing the captured/released microorganism. The culture plate may comprise LB agar.

The microorganisms may undergo functional tests, such as antimicrobial susceptibility tests and mechanism studies.

The method may comprise the step of separating the negatively-charged biological material from the microfilter by contacting or incubating the microfilter coated with the positively-charged degradable layer with a solution comprising a degradation agent. The degradation agent may result in the degradation of the positively-charged alginate gel layer, resulting in the separation of the negatively-charged biological material from the microfilter. In particular, the degradation agent may be adapted to remove the positive charge of the positively-charged degradable layer. Upon contact with the degradation agent, the positive charge of the positively-charged degradable layer may be removed, resulting in the dissolution of the positively-charged degradable layer, and the release of the negatively charged biological material captured thereon. The solution comprising a degradation agent may be an aqueous solution.

The degradation agent may be alginate lyase, ethylenediaminetetraacetic acid (EDTA), egtazic acid (EGTA), sodium citrate, nitrilotriacetic acid, n-hydroxyethylethylenediaminetriacetic acid (hEDTA) or combinations thereof. The degradation agent may additionally or alternatively be an organic acid such as oxalic acid, malic acid, rubeanic acid, citric acid or combinations thereof. The degradation agent may be EDTA or alginate lyase.

Advantageously, the method of separating biological material by removing of the positive charge from the positively-charged degradable layer allows for recovery of viable biological material for further functional studies. In particular, it was found that the degradation agent which removes the positive charge from the positively-charged degradable layer described herein does not adversely affect the structure and function of the recovered biological material. For example, microorganisms such as bacteria, fungi and viruses were found to be viable and culturable after recovery/release using the methods described herein. The microorganisms may or may not be released from the positively-charged degradable layer before culture. Where the microorganisms are released from the positively-charged degradable layer, a solution comprising the degradation agent and the released microorganisms may be contacted with an agar plate for subsequent culture of the microorganisms. Alternatively, the microfilter comprising the positively-charged degradable layer having the microorganisms captured thereon may be transferred to an agar plate for direct culture of the microorganisms, without first releasing or isolating the microorganisms from the microfilter coated with the positively-charged degradable layer. Advantageously, the use of a microfilter made from the biocompatible material may enable microorganisms to be directly cultured from the microfilter coated with the positively-charged degradable layer and the microorganisms captured thereon. Accordingly, the methods described herein advantageously allows for collection and recovery of viable biological material, which may then be used for downstream studies.

Alternatively, the method may comprise the step of removing the microfilter from the microfiltration device and incubating the removed microfilter with the degradation agent, whereby the microfilter comprises the positively-charged alginate gel layer with the negatively-charged biological material attracted thereon. Here, the degradation agent degrades or lyses the biological material (and may or may not degrade the microfilter).

The incubation may be carried out at a temperature and duration sufficient to degrade the positively-charged degradable layer and/or lyse the biological material. The temperature is not limited and may be in the range of about 25° C. to about 45° C., about 30° C. to about 45° C., about 35° C. to about 45° C., about 40° C. to about 45° C., about 25° C. to about 40° C., about 25° C. to about 35° C., or about 25° C. to about 30° C., for a duration of about 10 minutes to about 30 minutes, about 20 minutes to about 30 minutes or about 10 minutes to about 20 minutes. It should be appreciated that depending on the type of biological material and the downstream process needed, this then determines the temperature and duration required for the incubation. The temperature and duration can be selected to ensure viability of the biological material or be outside the viability limits of the biological material to aid in the degradation of the biological material.

The separated negatively-charged biological material may be collected in a collection tube containing an appropriate amount of a solution. The collection solution is of a smaller volume as compared to the initial sample so as to concentrate the recovered negatively-charged biological material.

Where the negatively-charged biological material are microorganisms, there may then be a further method for analysis, such as culturing the microorganisms (as mentioned above) or lysing the microorganisms. Where lysis of microorganisms is preferred, the method may comprise the steps of:
a) transferring the microorganisms into a lysis solution;
b) lysing the microorganisms in the solution of step a) to extract nucleic acids or proteins from said microorganisms;
c) centrifuging the solution in step b) for a duration to phase separate waste pellets and supernatant comprising the nucleic acid or proteins of the microorganisms; and
d) mixing the supernatant comprising the nucleic acids or proteins with a solution suitable for subsequent analysis.

In step (a), the microorganisms may already be recovered from the microfilter or may be still attracted to the positively-charged degradable layer coated on the microfilter.

The lysing solution may be adapted for the extraction of nucleic acids or proteins from the microorganism or pathogen. The lysing solution (depending on whether nucleic acids or proteins are to be extracted) may comprise a buffer solution with ionic salt and optionally a detergent. As an example, the lysis solution may be a buffer solution selected from Plant lysis buffer from Lucigen, QuickExtract™ DNA from Lucigen, One-step lysis buffer from Chai Bo, or Platinum direct lysis buffer from Invitrogen.

The lysing step b) may further comprise a step of i) homogenizing and ii) heating, where in homogenizing step i) and heating step ii) are optionally repeated one or more times in an alternating manner.

The homogenizing step can be carried using a vortex for a duration of about 5 seconds to about 60 seconds, about 10 seconds to about 60 seconds, about 20 seconds to about 60 seconds, about 40 seconds to about 60 seconds, about 5 seconds to about 40 seconds, about 5 seconds to about 20 seconds, about 5 seconds to about 10 seconds, or about 10 seconds to about 20 seconds. The homogenizing step may be carried out for a duration of about 15 seconds.

The heating step may be carried out at a temperature range of about 40° C. to about 100° C., about 60° C. to about 100° C., about 80° C. to about 100° C., about 40° C. to about 80° C., or about 40° C. to about 60° C., for a duration of about 2 minutes to about 10 mins, about 4 minutes to about 10 minutes, about 6 minutes to about 10 minutes, about 8 minutes to about 10 minutes, about 2 minutes to about 8 minutes, about 2 minutes to about 6 minutes, or about 2 minutes to about 4 minutes. It should be appreciated that depending on the type of lysis buffer used, this then determine the temperature and duration required for the heating step.

The first homogenizing step may be carried out by vortexing for a duration of 15 seconds and the first heating step may be performed at a temperature of 65° C. for about 6 minutes. The second homogenizing step may be carried out by vortexing for a duration of 15 seconds and the second heating step may be performed at a temperature of 98° C. for a duration of 2 minutes. The third homogenizing step may be carried out by vortexing for a duration of about 15 seconds.

The heating step may be conducted in a water bath or a dry bath/block heater.

The centrifugation step may be carried out at a relative centrifugal force of about 4 000×g to about 10 000×g, about 6 000×g to about 10 000×g, about 8 000×g to about 10 000×g, about 4 000×g to about 8 000×g, or about 4 000×g to about 10 000×g for a duration of about 10 seconds to about 60 seconds, about 20 seconds to about 60 seconds, about 40 seconds to about 60 seconds, about 10 seconds to about 40 seconds, or about 10 seconds to about 20 seconds. The centrifugation step may preferably be carried out a relative centrifugal force of about 5 000×g for a duration of 30 seconds.

The waste pellets may comprise the microfilter and unwanted biomaterial from the microorganisms (i.e., biomaterial other than nucleic acid) or the unwanted biomaterial only.

Using a micropipette, the supernatant comprising the nucleic acid or protein may be mixed with the solution in a concentration of about 0.01× to about 1.0×, about 0.05× to about 1.0×, about 0.1× to about 1.0×, about 0.2× to about 1.0×, about 0.4× to about 1.0×, about 0.6× to about 1.0×, about 0.8× to about 1.0×, about 0.01× to about 0.8×, about 0.01× to about 0.6×, about 0.01× to about 0.4×, about 0.01× to about 0.2×, about 0.01× to about 0.1×, or about 0.01× to about 0.05×.

The nucleic acid may be deoxyribonucleic acid, ribonucleic acid or a mixture thereof.

In step (d), depending on whether nucleic acid or protein is extracted, this determines the downstream analysis of the nucleic acid or protein. Where nucleic acid is extracted, the subsequent analysis may be PCR (which can be followed by sequencing as needed), electrophoresis, southern blotting, and so on. Where PCR is used, the solution is then a PCR master mix solution comprising an isothermal amplification buffer. As an example, the isothermal amplification buffer may be an isothermal buffer pack comprising of Tris-HCl, (NH₄)₂SO₄, KCl, MgSO₄, 0.1% Tween® 20 adjusted to pH 8.8 @25° C.

The master mix solution may comprise a nucleotide. The nucleotide may be deoxyribonucleotide (dNTP), ribonucleotide, or a combination thereof. The nucleotide may be deoxyribonucleotide (dNTP).

The master mix solution may comprise buffering salts. The buffering salts may be selected from the group comprising of magnesium sulfate, ammonium sulfate, sodium sulfate, potassium chloride and sodium chloride. The buffering salt may be magnesium sulfate (MgSO₄).

The master mix solution may comprise an amino acid. The amino acid is not limited and depends on the type of PCR used. As an example, the amino acid may be 2-aminoethanesulfonic acid (taurine).

The master mix solution may comprise a dye. The dye may have an excitation wavelength of about 600 nanometres to about 800 nanometres. The dye may be 680 dye (i.e. a dye with an excitation wavelength of about 680 nanometres).

The master mix solution may comprise a polymerase. The polymerase may be selected from the group comprising of DNA polymerase and RNA polymerase. The polymerase may be DNA polymerase, for example, Bst 2.0.

The master mix solution may comprise a primer set. The primer set may further comprise a forward inner primer, a backward inner primer, a forward outer primer, a backward outer primer, a forward loop primer and a backward loop primer.

Additionally, and alternatively, the master mix solution may comprise an isothermal amplification buffer, taurine, dNTP, magnesium sulfate (MgSO₄), Bst 2.0, 680 dye and a primer set.

The solution containing the nucleic acid and master mix may be loaded onto a polymerase chain reaction (PCR) chip for amplification. The amplification technique may be PCR or digital loop-mediated isothermal amplification (dLAMP). The amplification technique may be dLAMP. The amplification technique may be a warm start CRISPR/Cas-based dLAMP.

The loaded PCR chip may undergo dLAMP procedure.

Where protein is extracted, the protein may be subjected to protein analysis techniques such as western blotting, protein gel analysis, Edman degradation or mass spectrometry.

As the biological material is separated from the sample, the biological material can be deemed to be recovered from the sample and depending on the volume of the solution used to collect the recovered biological material, can then be deemed to be concentrated. Therefore, the microfiltration device (as described herein or as prepared by the method described herein) can also be one that is used to recover (and additionally concentrate) a biological material from a sample. The method of separating a biological material from a sample can then be one that is used to recover (and additionally concentrate) a biological material from the sample.

Exemplary, non-limiting embodiments of a kit comprising the microfiltration device will now be disclosed.

The microfiltration device is as defined above and may comprise the microfilter with the positively-charged degradable layer thereon, a sampling zone which receives the sample and directs the sample to the positively-charged degradable layer coated on the microfilter, and a collection zone located on the opposite side of the microfilter for collecting the filtered sample or filtrate. For example, the sampling zone may be the area above the positively-charged degradable layer, while the collection zone may be the area below the microfilter.

The kit may further comprise a component for receiving a sample, for example, the component may be a sampling tube. The sampling component may further comprise a rubber O-ring to seal the sampling tube against the sampling zone of the microfiltration device.

The kit may further comprise a component for drainage of a sample, for example, the component may be a drainage tube. The drainage tube may be connected to the collection zone. The drainage component may further comprise a component for means of locking the sampling component and the drainage component with the microfiltration device sealed in between.

The kit may further comprise a housing container to hold said locked sampling and drainage components. The housing container may be a tube, for example, a Falcon tube.

Exemplary, non-limiting embodiments of a microfluidic capture device comprising a positively-charged degradable layer will now be disclosed.

The microfluidic capture device may comprise a positively-charged degradable layer. The positively-charged degradable layer may be a positively-charged alginate gel layer prepared according to the method described herein which may be coated onto, but not limited to, filters, filter membranes or non-filter structures.

The coated filters, filter membranes and non-filter structures may be incorporated into a device for microfluidic capturing of analytes from a sample.

The microfluidic capture device may separate biological material from large-volume samples with complex background and concentrate the separated biological material to be compatible with downstream analysis techniques.

Exemplary, non-limiting embodiments of a microfilter will now be disclosed.

The microfilter comprises a positively-charged degradable layer coated thereon. The positively-charged degradable layer may be a positively-charged alginate gel layer. The microfilter and positively-charged degradable layer are as described herein.

Exemplary, non-limiting embodiments of a kit-of-parts will now be disclosed.

The kit-of-parts comprises a microfilter having a positively-charged layer thereon, a sampling zone and a collection zone. The positively-charged degradable layer may be a positively-charged alginate gel layer. In use, the microfilter may be connected or coupled with the sampling zone on one side of the microfilter and the collection zone on the opposite side of the microfilter to form a microfiltration device. The microfiltration device, sampling zone and collection zone are as described herein.

The kit-of-parts may further comprise a sampling tube or sampling container and/or a drainage tube.

Advantageously, the microfiltration device as described herein provides an easier method to separate biological material from a sample without the need for huge facilities or instruments (e.g., centrifuge) in a rapid manner (taking less than 30 minutes for >100 mL crude samples). The microfiltration device as described herein allows for higher enrichment factors when separating biological material from a sample, which improves compatibility with various downstream methods.

Further advantageously, the microfiltration device as described herein is both portable and friendly for single usage, hence this makes it friendly for usage in point-of-care testing scenarios even in remote or resource-limited areas.

### DETAILED DESCRIPTION OF DRAWINGS

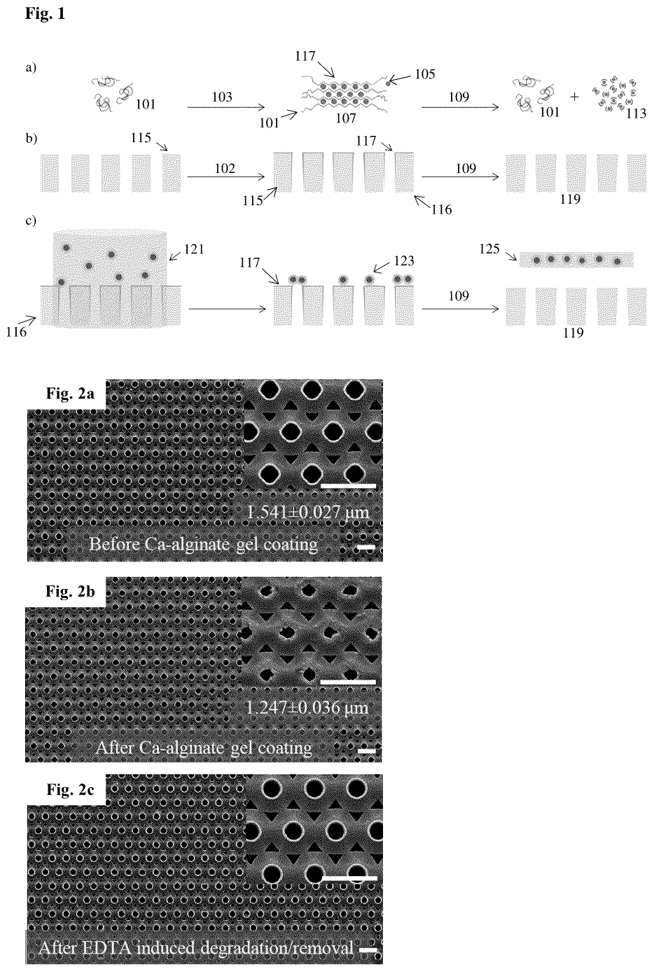

Referring to FIG. 1, the working principle of a microfiltration device comprising a microfilter coated with a positively-charged degradable layer and its use in a method of for separating biological material from a sample will now be explained. In row a) of FIG. 1, a positively-charged degradable layer 117 may be prepared from a polymer substrate solution 101 and a cross-linking agent 103. The crosslinking agent 103 may comprise cations 105 which facilitate the formation of crosslinks 107 in the polymer substrate 101, and impart a net positive charge when forming into the positively-charged degradable layer 117. A mixture 102 of the cross-linking agent 103 and the polymer substrate 101 may be coated onto a microfilter 115 to provide a positively-charged degradable layer 117 on the surface of the microfilter 115 making up a microfiltration device 116, as shown in row (b) of FIG. 1. A degradation agent 109 which may disrupt the crosslinks 107 in the positively-charged degradable layer 117 may be used to dissolve or degrade the positively-charged degradable layer 117. For example, the degradation agent 109 may form a complex 113 with the cations 105 to dissolve or degrade the positively-charged degradable layer 117, resulting in the formation of the complex 113 and the polymer substrate solution 101. The dissolution or degradation of the positively-charged degradable layer 117 also results in the loss of the surface charge from microfilter 119. Referring to row c) of FIG. 1, when used for the separation of biological material, a sample 121 containing biological material may be passed through the microfiltration device 106, to capture biological material on the positively-charged degradable layer 117. The captured biological material 123 may be recovered by contacting the positively-charged degradable layer 117 with a degradation agent 109, to dissolve or degrade the positively-charged degradable layer 117 and release the captured biological material. This may allow the recovery of concentrated biological material 125 for downstream processing or detection. The resultant microfilter 119 may then be reused (as microfilter 115) and be coated with the positively-charged degradable layer 117 again.

### EXAMPLES

### Example 1: Method of Preparing Microfiltration Device

Herein, a Parylene C microfilter was first purchased from Hangzhou Branemagic Medical Technology Co. Ltd. (of Hangzhou, China) with a thickness of about 5 μm and a high porosity of >14%. A SEM image of the surface of the Parylene C microfilter is shown in FIG. 2a.

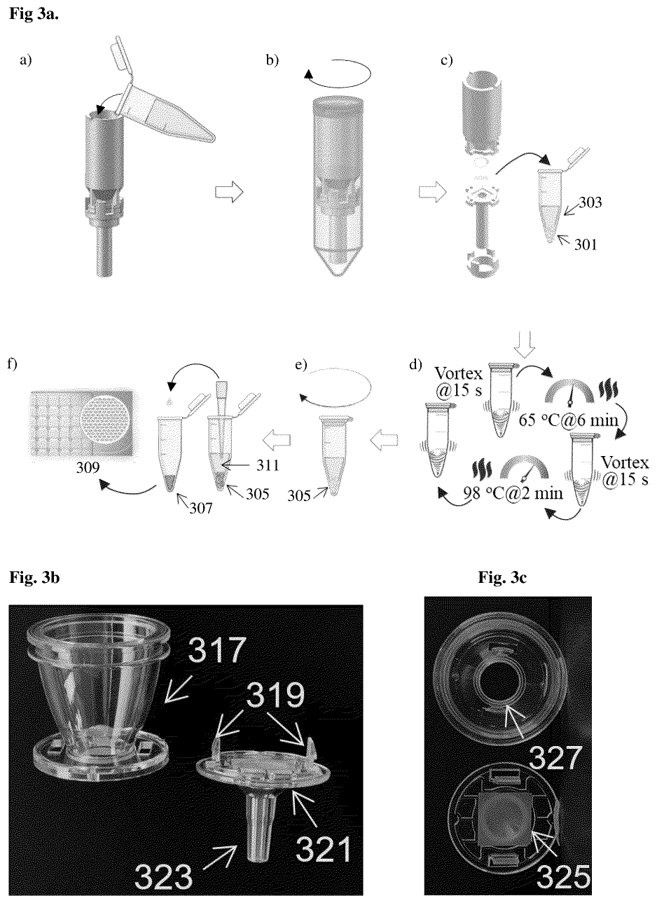

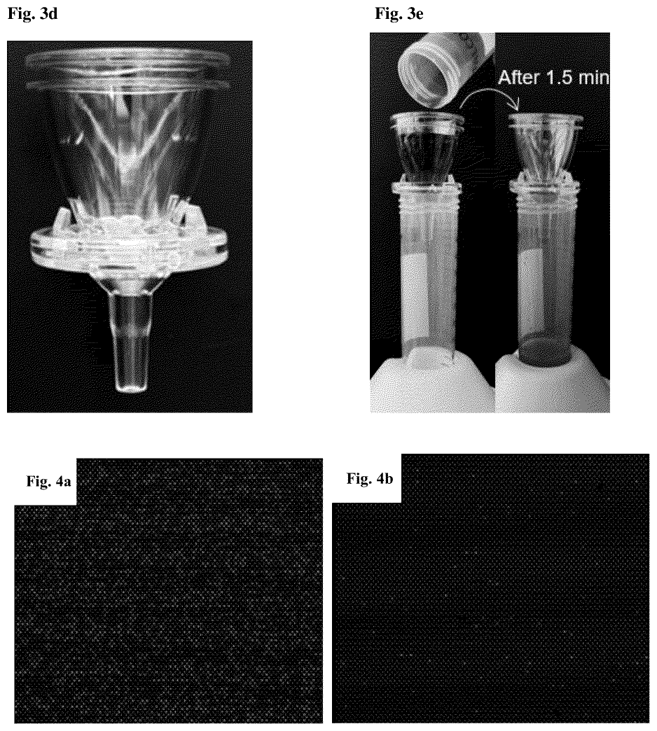

An alginate gel layer was then subsequently prepared from alginic acid sodium salt and calcium chloride (CaCl₂)) (product no. A1112 and C5670 from Sigma Aldrich). 0.4% of alginic acid sodium salt and 10 mM of CaCl₂) solution was prepared and a volume of 0.2 mL to 0.8 mL of the solution was added to the microfilter in a spin coater (obtained from POLOS) at 500 rotations per minute for 10 seconds, followed by 2 000 rotations per minute for 60 seconds to produce a first Ca-alginate gel layer. A second Ca-alginate gel layer was then added, following the method as just described herein, on top of the first Ca-alginate gel layer to obtain a total thickness of the various layers of 0.2 to 2 micrometres. Referring to row a) of FIG. 1, the positively-charged degradable layer (117, in the form of the Ca-alginate gel) was therefore prepared from a polymer substrate solution (101, in the form of the alginic acid sodium salt) and a cross-linking agent (103, in the form of the CaCl₂) solution) and subsequently coated onto the surface of a microfilter (115, in the form of the Parylene microfilter). As shown in FIG. 3b to FIG. 3d and in row b) of FIG. 1, the formed microfiltration device 106 is then the microfilter (115, the Parylene microfilter) coated with the positively-charged degradable layer (117, the Ca-alginate gel), which is placed over an opening 327 provided on support 321 and is secured to the sampling container 317 using securing means 319. The support is detachable from the sampling container 317 and comprises a drainage tube 323 to direct the flow of the filtrate to a collection container. The surface of the microfilter coated with a Ca-alginate gel coating was studied by SEM, as shown in FIG. 2b.

### Example 2: Results of Bacteria Capture and dLAMP Detection

The method of FIG. 3 was followed here. With reference to step a) of FIG. 3a, and as illustrated in FIG. 3e, a sample having a volume of about 1 to 10 mL comprising biological material was introduced to the microfiltration device made according to Example 1 (pore size of about 1.2 μm, porosity of 9%). The biological material in the sample included S. aureus (Sa.6538), K. pneumoniae or P. aeruginosa (Pa. 9027) bacteria (purchased from the American Type Culture Collection). In step b), the sample comprising the biological material was passed through the microfiltration device using ultrafiltration, by centrifuging the microfiltration device at a speed of 500×g for 1 minute to recover the biological material on the positively-charge alginate gel layer. The microfilter 301 comprising the isolated bacteria on the alginate gel layer was removed from the microfiltration device and transferred to a 1.5 mL microcentrifuge tube containing 0.06 mL of lysis solution 303, as shown in step c). The lysis solution was Plant lysis buffer obtained from Lucigen. SEM images of the microfilter coated with an alginate gel layer after filtration of the sample show the capture of S. aureus on alginate gel layer of the microfilter (FIG. 6). In step d), the tube containing the microfilter and lysis solution was subjected to a homogenising and heating cycle to induce lysis of the bacteria. The microcentrifuge tube was initially vortexed at 1 000 rpm for 15 seconds (vortex machine from ThermoFischer Scientific), followed by a first heating at 65° C. for 6 minutes, followed by a second vortex at 1 000 rpm for 15 seconds and a second heating at 98° C. for 2 minutes. Afterwards, in step e), the solution was centrifuged at 5 000×g for 30 seconds (centrifuge from ThermoFisher Scientific) to separate the lysate into waste pellet 305 and supernatant 311, which contains nucleic acids.

In step f) of FIG. 3, 8 μL of supernatant 311 comprising nucleic acids was pipetted into 32 μL of master mix solution 307 to obtain 0.2× concentration (total volume of 40 μL). It should be appreciated that, the total volume may be varied based on the dPCR machine being used. In addition, the volume ratio of the supernatant to the master mix solution may vary according to the dPCR procedure which is adopted. 40 μL of the resulting solution was then transferred into a well (triplicates per sample) of PCR chip 309 (obtained from Qiagen) and subsequently analysed via a digital loop-mediated isothermal amplification (dLAMP) procedure. The master mix solution was prepared according to Table 1 below.

TABLE 1
Reaction system of dLAMP

| No. | Reagent | | Concentration | |
|---|---|---|---|---|
| 1 | Isothermal amplification buffer | | 1× | |
| 2 | Taurine | | 50 | mM |
| 3 | dNTPs | | 1.4 | mM |
| 4 | MgSO₄ | | 6 | mM |
| 5 | Bst 2.0 | | 2 | mM |
| 6 | 680 dye | | 10 | μM |
| 7 | Primer | Forward Inner Primer | 1.6 | μM |
| | set | Backward Inner Primer | 1.6 | μM |
| | | Forward Outer Primer | 0.2 | μM |
| | | Backward Outer Primer | 0.2 | μM |
| | | Forward Loop Primer | 0.4 | μM |
| | | Backward Loop Primer | 0.4 | μM |

| | | |
|---|---|---|
| 8 | Bacteria lysate | 0.2× |

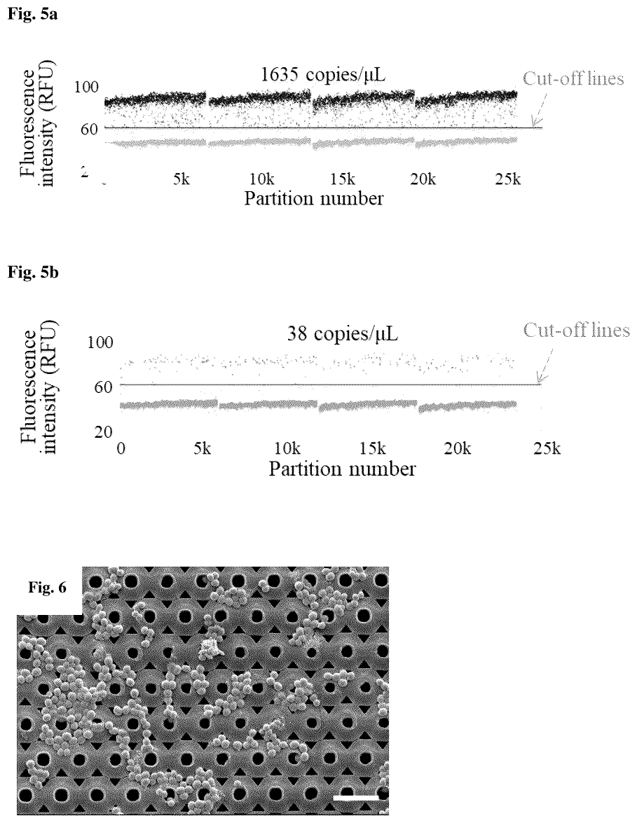

For comparison, a traditional centrifugation technique was used to concentrate a sample containing S. aureus (Sa.6538) bacteria. The obtained pellet was subjected to the lysis procedure of steps c) to f) in the method of FIG. 3, for dLAMP analysis. It can be seen that using the electrostatic microfiltration (EM) method of bacteria capture as described herein, there is a higher intensity of fluorescence distribution for the EM sample (FIG. 4a) as compared to the centrifugated (C) sample (FIG. 4b). Each fluorescent dot represents a positive signal of a droplet/partition in the dLAMP detection analysis, hence, the higher ratio/percentage of fluorescent dots in FIG. 4a indicates better recovery and concentration of the microorganisms using the microfiltration device. Further, the partition scatter plot for the EM sample (FIG. 5a) shows 1635 copies/μL as compared to 38 copies/μL for the C sample (FIG. 5b), thus showing that the method of microorganism separation using the microfiltration device as described herein affords a much higher enrichment factor.

The enrichment factor is calculated by the ratio of input volume and recovered volume after separation; in this example, the input volume of 10 mL was reduced to 60 μL, thus giving an enrichment factor of ~166×.

The results of bacteria capture using the method as described herein versus traditional centrifugation is shown in Table 2 below.

TABLE 2
Results of bacteria capture

| | CFU | | Capture | dLAMP signals (copies/μL) | |
|---|---|---|---|---|---|
| | count | | efficiency | | Normalized |
| No. | (×10⁴) | | (%) | Original | (for 10⁴ CFU) |
| 1 | 1.25 | EM | 99.9 | 1635 | 1310 |
| | | C | 21.1 | 38 | 30 |
| 2 | 1.09 | EM | 63.6 | 1316 | 1210 |
| | | C | 16.9 | 49 | 45 |
| 3 | 1.46 | EM | 99.9 | 512 | 350 |
| | | C | 15.8 | 12 | 8 |
| Ave ± | 1.27 ± | EM | 87.8 ± 21.0 | 1155 ± 580 | 957 ± 528 |
| SD | 0.19 | C | 17.9 ± 2.8 | 33 ± 19 | 28 ± 19 |

The capture efficiency was calculated as follows:

$$Capture efficiency = \frac{\begin{matrix}Total number of bacteria in the starting sample \\ ( Concentration of bacteria in \frac{CFU}{mL} \times sample volume ( mL ) ) - \\ Number of bacteria in the filtrate \\ ( Concentration of bacteria in \frac{CFU}{mL} \times sample volume ( mL ) )\end{matrix}}{\begin{matrix}Total number of bacteria in the starting sample \\ ( Concentration of bacteria in \frac{CFU}{mL} \times sample volume ( mL ) )\end{matrix}} \times 100 \%$$

As seen from Table 2, the average capture efficiency of the electrostatic microfiltration is 87.8±21.0% for an abundance of ~10⁴ CFU/mL (with S. aureus 6538 as an example), which is much higher than that of centrifugation (10 000×g@10 min) at an average capture efficiency of 17.9±2.8%. Further, the high-fold (>20×) enrichment (from 1 mL down to 50 μL) of the electrostatic microfiltration-based sample preparation improved the dLAMP detection readout by about 37±9 times (from 27.78±19 to 957±528 copies/μL, normalization of 10⁴ CFU, n=3) compared to the centrifugation-based process.

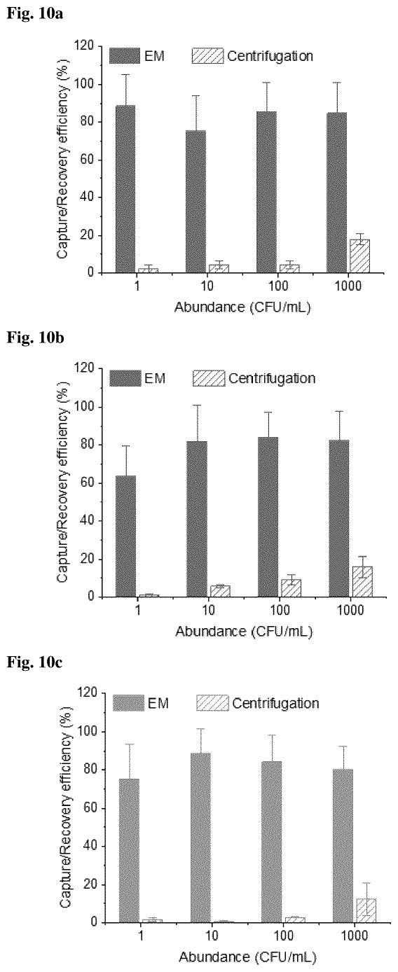

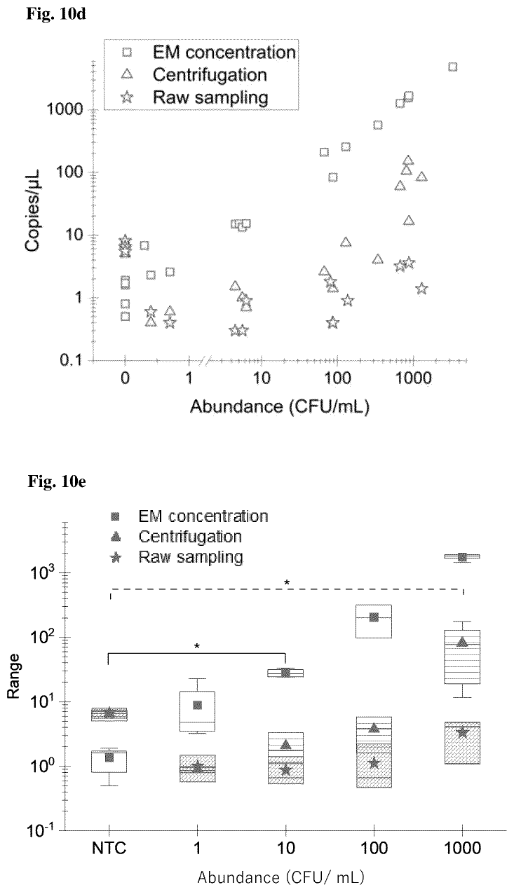

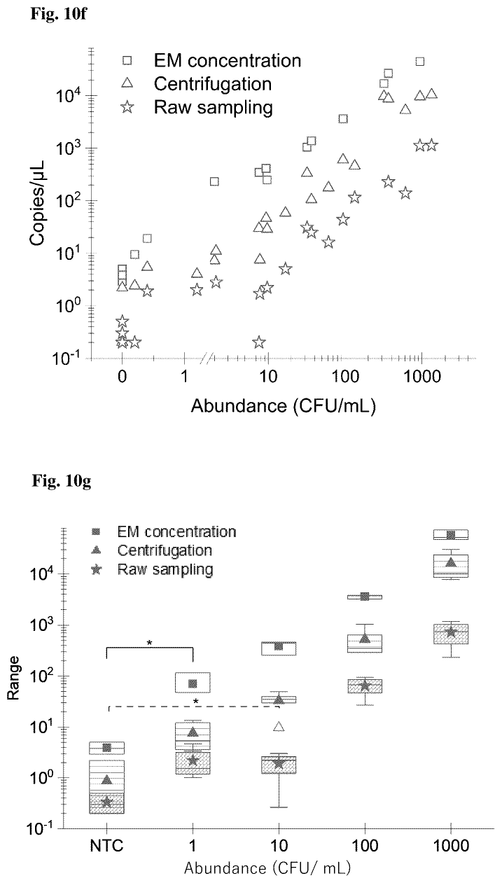

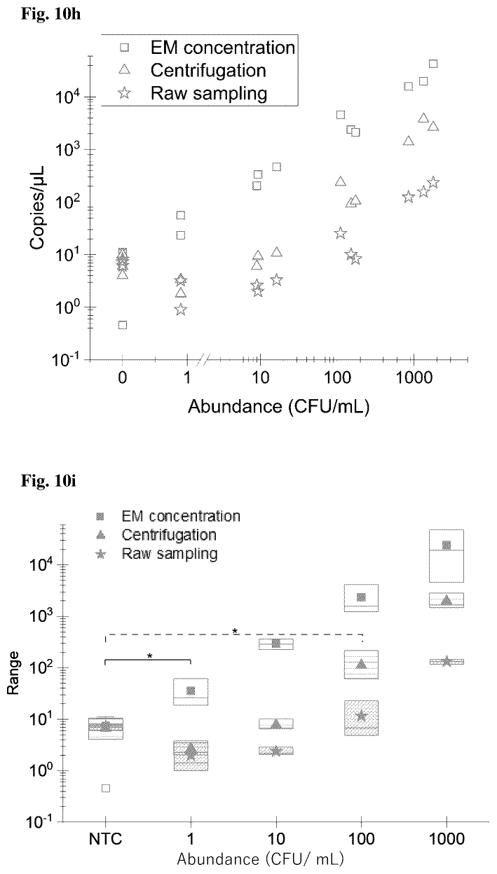

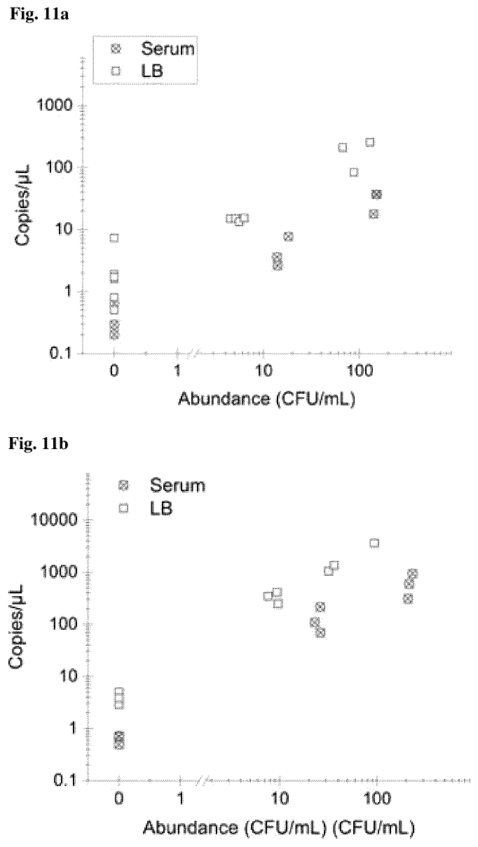

To further investigate the capture efficiency of the microfiltration device, the procedure above was repeated for 10 mL samples containing S. aureus, K. pneumoniae or P. aeruginosa at concentrations of 1 CFU/mL, 10 CFU/mL, 100 CFU/mL or 1000 CFU/mL. For comparison, the capture or recovery of bacteria by was also carried out by centrifuging samples comprising S. aureus, K. pneumoniae or P. aeruginosa at the concentrations above, at a speed of 10,000× g for a duration of 10 minutes. The capture/recovery efficiency of the microfiltration device for the capture of S. aureus is shown in FIG. 10a, while the capture/recovery efficiencies of the microfiltration device for K. pneumoniae is shown in FIG. 10b. FIG. 10c shows the capture/recovery efficiency of the microfiltration device for the capture of P. aeruginosa.

A graphical plot of the dLAMP signals of the bacterial DNA of recovered S. aureus after degradation of the positively charged degradable layer is shown in FIG. 10d and FIG. 10e. The obtained dLAMP signals of the bacterial DNA of recovered K. pneumoniae recovered from microfilter is shown in FIG. 10f and FIG. 10g, while the dLAMP signals of bacterial DNA of P. aeruginosa recovered from the microfilter is shown in FIG. 10h and FIG. 10i. The dLAMP signals of the bacterial DNA of S. aureus, K. pneumoniae or P. aeruginosa in the sample prior to filtration (raw sampling) is also shown in FIG. 10d to FIG. 10i (indicated with the star markers ★ and ☆). For comparison, the dLAMP signals of the bacterial DNA of S. aureus, K. pneumoniae or P. aeruginosa in the pellet recovered by conventional centrifugation methods is also shown in FIG. 10d to FIG. 10i (indicated with ▴ and Δ)

The developed microfiltration device demonstrated superior capture efficiency for various bacteria (3 out of 6 ESKAPE pathogens including S. aureus, K. pneumoniae and P. aeruginosa tested as typical examples) even at low concentrations of microorganisms (down to 1 CFU/mL). For the cases of 1000 CFU/mL and 1 CFU/mL, the method of separating the biological method described herein can achieve more than 5× and more than 40× higher capture efficiency compared to centrifugation, respectively (FIG. 10a to FIG. 10i).

The electrostatic microfiltration-based sample preparation interfacing digital PCR (dPCR) can push forwards the limit-of-detection (LOD) to about 100× (for S. aureus, and P. aeruginosa, FIG. 10d, FIG. 10e, FIG. 10h and FIG. 10i), and 10× (for K. pneumoniae, FIG. 10f and FIG. 10g) lower compared to conventional centrifugation methods. The LOD was about 1000× lower for the capture of S. aureus and P. aeruginosa, and 100× lower for capture of K. pneumoniae, as compared to raw sampling.

### Example 3. Capture and Release of Viable Biological Material

The microfiltration device of Example 1 was used for the separation of biological material. A sample comprising K. pneumoniae at a concentration of 1×10⁶ CFU/mL was prepared for separation using the microfiltration device. With reference to row c) of FIG. 1, a sample containing biological material 121 (a sample containing S. aureus at 1×10⁶ CFU/mL, a sample containing K. pneumoniae at 1×10⁶ CFU/mL or a sample containing P. aeruginosa at 1×10⁶ CFU/mL) was passed through the microfiltration device 106 where the microfilter 115 coated with the positively-charged degradable layer (117, in the form of the Ca-alginate gel) has a pore size of about 1.2 μm, porosity of 9%.

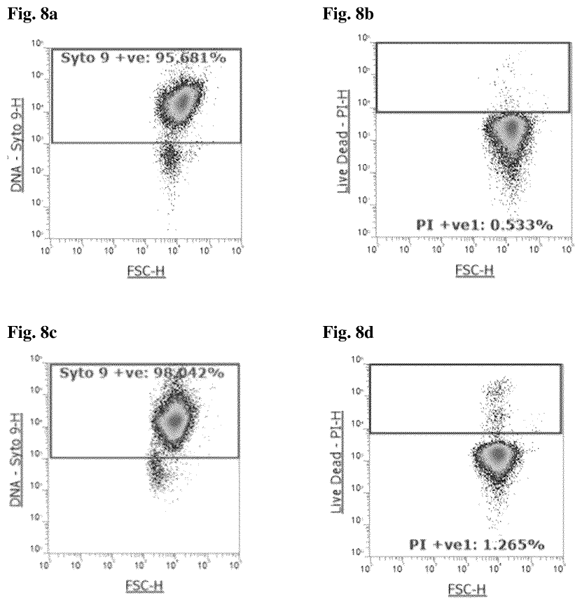

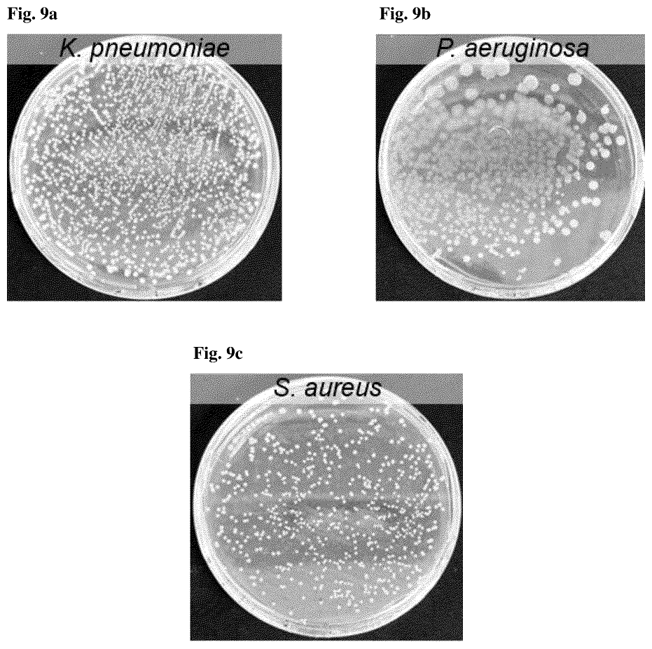

The microfilter comprising the biological material 123 (e.g. K. pneumoniae) captured by the Ca-alginate gel was incubated with a solution of a degradation agent 109 (EDTA at a concentration of 10 mM or alginate lyase at a concentration of 0.1 U/mL). The degradation product was removed along with the captured biological material 123, leaving the microfilter 119 without a surface charge. A SEM image of the microfilter after removal of the Ca-alginate gel is shown in FIG. 2c. An aliquot of the solution of the degradation agent, the degraded positively charged degradable layer and the captured biological material (K. pneumoniae) was removed and applied to an agar plate for subsequent culture. The remaining solution comprising the captured biological material was used for flow cytometry studies to determine the viability of the captured biological material. A photograph of well-grown colonies of K. pneumoniae recovered from the microfiltration device is shown in FIG. 9a. Flow cytometry studies indicated that the microorganisms recovered after capture on the positively-charged degradable layer were highly viable (FIG. 8c and FIG. 8d) and were comparable to the control samples (FIG. 8a and FIG. 8b).

The steps above were repeated for the recovery of microorganisms from a sample of S. aureus provided at a concentration of 1×10⁶ CFU/mL and a sample of P. aeruginosa provided at a concentration of 1×10⁶ CFU/mL. The captured microorganisms were released using the degradation agent (EDTA at a concentration of 10 mM or alginate lyase at a concentration of 0.1 U/mL) and the release solution comprising the degradation agent and the captured microorganisms were applied to an agar plate for further culture. FIG. 9b shows the cultured colonies of recovered P. aeruginosa and FIG. 9c shows the cultured colonies of recovered S. aureus.

### Example 4. Detection of Microorganisms in a Sample Containing a Mixture of Microorganisms

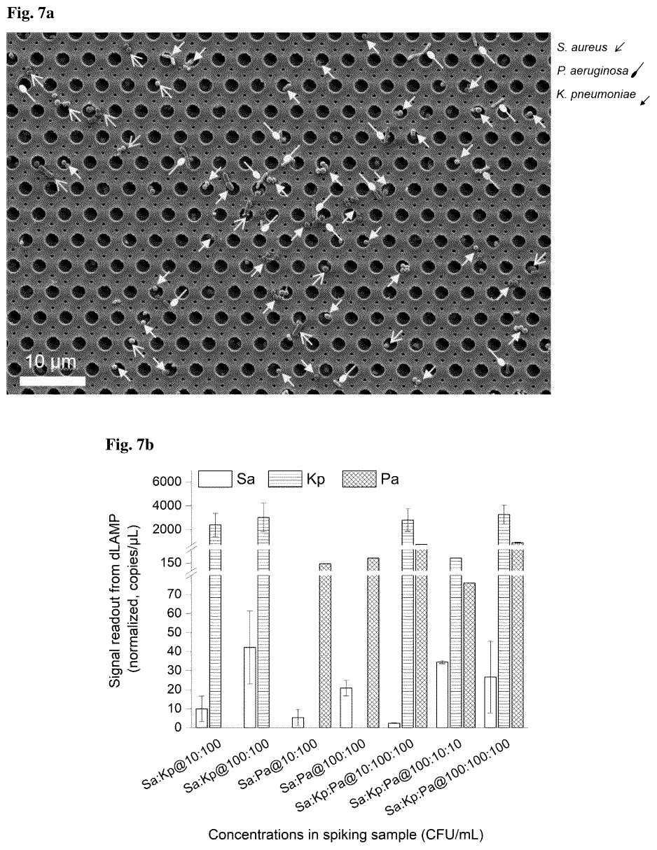

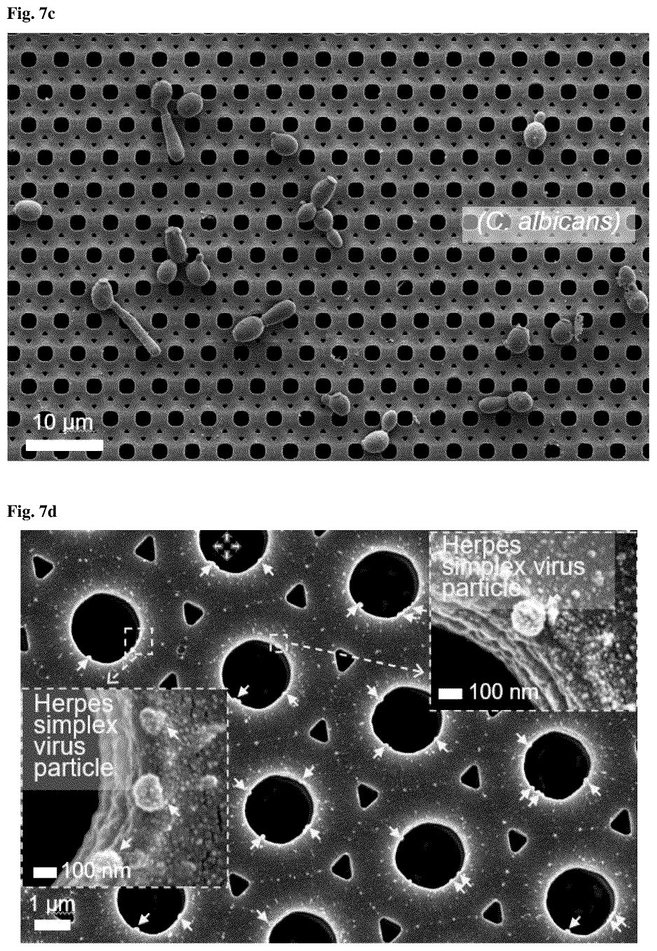

The microfiltration device of Example 1 (pore size of about 1.2 μm, porosity of ≥9%) was used for the filtration of samples comprising a mixture of biological material. A sample comprising a mixture of 1×10⁶ CFU/mL S. aureus, 1×10⁶ CFU/mL K. pneumoniae and 1×10⁶ CFU/mL P. aeruginosa was filtered through the microfilter of example 1. A SEM image of the microfilter coated with the positively-charged degradable layer showed the capture of the bacteria (FIG. 7a) on the microfilter coated with positively-charged degradable layer. The captured S. aureus bacteria is indicated with the arrow [symbol] , the captured P. aeruginosa bacteria is indicated with the arrow [symbol] , while the captured K. pneumoniae bacteria is indicated with [symbol] .

With reference to row c) of FIG. 1, the microfilter comprising the biological material 123 captured by the Ca-alginate gel was then incubated with a solution of a degradation agent 109 (EDTA at a concentration of 10 mM or alginate lyase at a concentration of 0.1 U/mL). The degraded product was removed along with the captured biological material 123, leaving the microfilter 119 without a surface charge. A SEM image of the microfilter after removal of the Ca-alginate gel is shown in FIG. 2c. The solution of the captured biological material and the degradation agent was then incubated with a master mix of comprising the reagents listed in Table 3. The detection of the bacterial DNA was carried out by dLAMP.

TABLE 3
Reaction system for detection of multiple bacteria

| Reagents | Final concentration | |
|---|---|---|
| Isothermal Amplification Buffer (NEB, B0537S) | 1x | |
| Taurine | 50 | μM |
| Deoxynucleoside Triphosphate, PCR grade (Roche, 03622614001) | 1.4 | mM |
| Magnesium Sulfate (MgSO₄) solution (NEB, B1003S) | 6 | mM |
| WarmStart RTx Reverse Transcriptase (NEB, M0380L) | 0.3 | U/ul |
| RNase Inhibitor, Murine (NEB, M0314L) | 1 | U/ul |
| Bst 2.0 WarmStart DNA Polymerase (NEB, M0538M) | 1 | U/ul |

| | |
|---|---|
| Reference dye (0.01 nm Cyanine 680SE) | |
| 3 Bacteria specific primer mix | Forward Inner Primer & Backward |
| | Inner Primer (1.6 μM for each) |
| | Forward Outer Primer & Backward |
| | Outer Primer (0.2 μM for each) |
| | Forward Loop Primer & Backward |
| | Loop Primer (0.4 μM for each) |
| 3 Bacteria specific fluorescent probe | 0.2 μM for each |

The dLAMP readout for the detection of the bacterial DNA of S. aureus, K. pneumoniae and P. aeruginosa is provided in FIG. 7b.

The steps above were repeated using samples comprising: i) a mixture of 10 CFU/mL S. aureus and 100 CFU/mL of K. pneumoniae, ii) a mixture of 100 CFU/mL S. aureus and 100 CFU/mL of K. pneumoniae, iii) a mixture of 10 CFU/mL S. aureus and 100 CFU/mL of P. aeruginosa, iii) a mixture of 100 CFU/mL S. aureus and 100 CFU/mL of P. aeruginosa, iv) a mixture of 10 CFU/mL S. aureus, 100 CFU/mL of K. pneumoniae and 100 CFU/mL of P. aeruginosa, v) a mixture of 100 CFU/mL S. aureus, 10 CFU/mL of K. pneumoniae and 10 CFU/mL of P. aeruginosa and v) a mixture of 100 CFU/mL S. aureus, 100 CFU/mL of K. pneumoniae and 100 CFU/mL of P. aeruginosa. The dLAMP signals of the bacterial DNA are as indicated in FIG. 7b. This demonstrates the versatility of the microfiltration method described herein for the capture and downstream detection of microorganisms, even when provided in a mixture.

### Example 5. Capture of Bacteria from Samples with High Background

High-concentration protein backgrounds are typically present in liquid samples of biological material. Accordingly, the capture of bacteria from samples spiked with fetal bovine serum was carried out using the microfiltration device described herein, interfaced with dPCR.

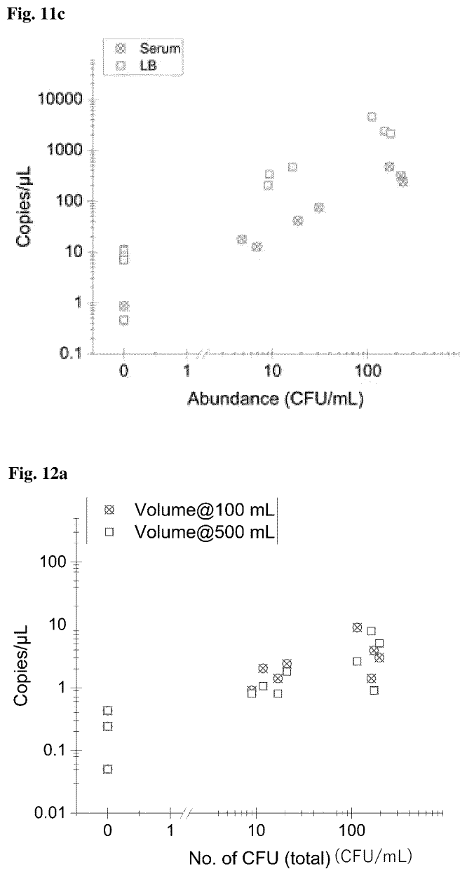

A 10 mL sample comprising a S. aureus at a concentration of 10 and 100 CFU/mL in Luria Broth (LB) was spiked with 10 mL of fetal bovine serum (FBS). For comparison, 10 mL samples comprising S. aureus in LB only (i.e. without serum) were prepared. The samples were filtered using the microfilter prepared in Example 1, and the subsequent detection of the captured bacteria was carried out according to the dLAMP procedure described in Example 2 (steps a to f of FIG. 3). The steps above were repeated for samples comprising K. pneumoniae, and samples comprising P. aeruginosa. The dLAMP signals of the bacterial DNA of recovered S. aureus is shown in FIG. 11a, FIG. 11b shows the dLAMP signals of bacterial DNA of recovered K. pneumoniae, while FIG. 11c shows the dLAMP signals of bacterial DNA of P. aeruginosa. The results show that the LOD of the bacteria recovered from samples spiked with serum (round marker ⊗) is comparable to that of pure Luria Broth (LB) (square marker □), as indicated in FIG. 11a to FIG. 11c. This is consistent for all 3 tested bacteria (FIG. 11a to FIG. 11c).

### Example 6. Capture of Bacteria from Samples of Large Volumes

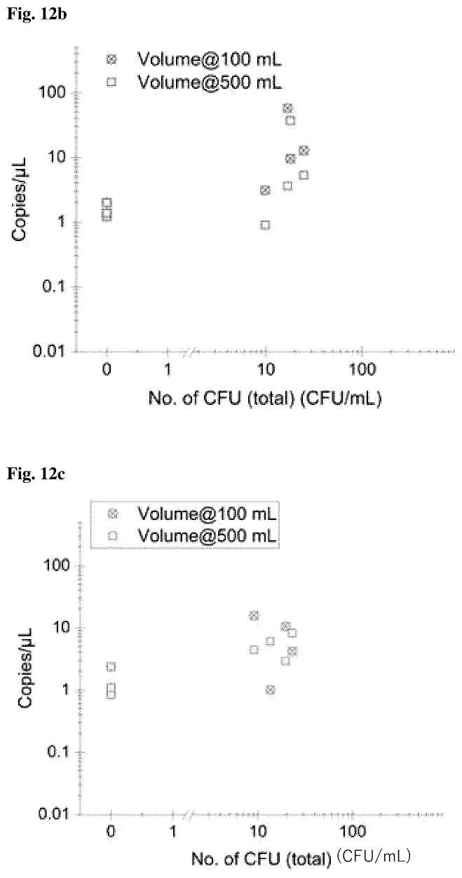

To investigate the throughput of the microfiltration device, large volume samples containing bacteria were filtered through the microfilter prepared in Example 1. A 100 mL and 500 mL sample of water containing S. aureus at concentrations of 10 CFU/mL and 100 CFU/mL were passed through the microfiltration device of Example 1. The separation of the biological material from the sample and the subsequent detection of the captured biological material was carried out according to the methods of Example 2 (steps a to f of FIG. 3). The procedure above was repeated for 100 mL and 500 mL samples comprising K. pneumoniae or P. aeruginosa at concentrations of 10 CFU/mL and 100 CFU/mL. The dLAMP signals of recovered S. aureus is shown in FIG. 12a, while FIG. 12b and FIG. 12c show the digital signals of K. pneumoniae and P. aeruginosa recovered from the 100 mL (round marker ⊗) and 500 mL (square marker □) samples

The method of separating the biological material with the microfiltration device described herein advantageously allows high-volume throughput due to the high porosity of the microfilter coated with the positively-charged degradation layer, which ensures the feasibility of processing ultra-large volume samples. The results show that the LOD can go down to 10 CFU for a 500 mL sample (i.e., 1 CFU/50 mL), for the recovery of bacteria. The concentration of the bacteria sample from 500 mL to 60 μL thus provided an enrichment factor of about 8333.

### Example 7. Capture of Viruses or Fungi

To demonstrate the universal applicability of the microfiltration device for the separation of microorganisms, a sample comprising C. albicans at a concentration of 1×10⁶ CFU/mL and a sample comprising Herpes Simplex virus provided at a concentration of 1×10⁶ PFU/mL were prepared and filtered through the microfiltration device of example 1. An SEM image of the captured C. albicans on the microfilter coated with the positively-charged degradable layer is shown in FIG. 7c, while FIG. 7d shows a SEM image of the captured HSV on the microfilter coated with the positively-charged degradable layer.

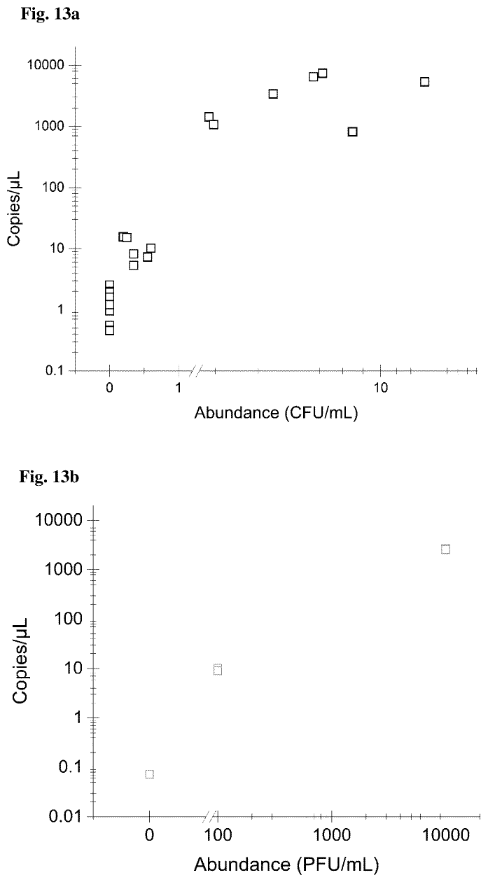

Further, 10 mL of a spiked Brain Heart Infusion (BHI) broth containing C. albicans at a concentration of 1 CFU/mL and 10 CFU/mL and 10 mL of spent media of mimic cell therapy product containing Herpes Simplex Virus (HSV) at a concentration of 100 and 10000 PFU/mL were tested. The samples were filtered using the microfilter prepared in Example 1. Detection of the fungi was carried out based on the procedure of Example 2, using the dLAMP system of Table 2, while the detection of HSV was carried out based on the procedure of Example 2, using the dPCR reaction system of Table 4 below. The dLAMP signals of the fungal DNA of C. albicans recovered from the microfilter is shown in FIG. 13a, while the dPCR signals of HSV recovered from the microfilter is shown in FIG. 13b.

TABLE 4
Reaction system for detection of HSV

| | |
|---|---|
| Reagent | Concentration |

| 4× QIAcuity Probe PCR Master Mix (Qiagen) | 1× | |
|---|---|---|
| Forward Primer | 800 | nM |
| Reverse Primer | 800 | nM |
| HSV fluorescent probe | 400 | nM |

| | |
|---|---|
| HSV (virus) lysate | 0.2× |

The results demonstrated that the LOD for C. albicans in 10 mL samples can go down to 1 CFU/mL (FIG. 13a) and LOD for HSV in 10 mL samples can go down to at least 100 PFU/mL (FIG. 13b). The dPCR signals for 100 PFU/mL samples are higher than 10 copies/μL (1 order difference from NTC samples), and LOD can be expected to go down at least 10 PFU/mL.

### INDUSTRIAL APPLICABILITY

The microfiltration device and the method of separating biological material may also be used as a sample preparation method which may be carried out prior to the analysis or detection of the biological material. For example, the microfiltration device and the method of separating biological material from a sample, as described herein may be used for the detection and surveillance of the level of biological materials in a collected sample, which may be a sample collected from the environment. The microfiltration device and the method of separating biological material described herein may be interfaced with various downstream analysis methods and techniques, and may even allow the recovery of viable biological material for further studies. The microfiltration device and the method of separating biological material may also result in the recovery of the biological material from the sample, usually with an increase in the concentration of the recovered biological material.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A microfiltration device comprising a microfilter coated with a positively-charged degradable layer.

2. The microfiltration device of claim 1, wherein the microfilter is made from a biocompatible material.

3. The microfiltration device of claim 2, wherein the biocompatible material is a biocompatible polymer selected from the group consisting of Parylene C, Parylene N, Parylene D, Parylene AF-4 and combinations thereof.

4. The microfiltration device of claim 1, wherein the positively charged degradable layer comprises a polymer substrate, and a cation.

5. The microfiltration device of claim 4, wherein the polymer substrate is gelatin, agar, iota carrageenan gel, alginate gel, Gellan gum, Xanthan gum, Guar gum, Gum arabic or Acacia gum; and the cation is selected from the group consisting of calcium (II) (Ca²⁺), strontium (II) (Sr²⁺), barium (II) (Ba²⁺), iron (II) (Fe²⁺), copper (II) (Cu²⁺), zinc (II) (Zn²⁺), potassium (K⁺), sodium (Na⁺), lithium (Li⁺), copper (I) (Cu⁺) and mixtures thereof.

6. (canceled)

7. The microfiltration device of claim 4, wherein the polymer substrate is alginate gel and the cation is calcium (II) (Ca²⁺).

8. The microfiltration device of claim 1, wherein the microfilter coated with the positively-charged degradable layer has a porosity of 9% or more.

9. The microfiltration device of claim 1, wherein the positively charged degradable layer has a thickness of about 0.2 μm to 2 μm and/or wherein the microfilter coated with the positively charged degradable layer comprises pores with an average diameter of about 1 μm to about 10 μm.

10. A method of separating a biological material from a sample, comprising the step of passing the sample comprising the biological material through a microfiltration device of claim 1, whereby the biological material is captured on the positively-charged degradable layer on the microfilter.

11. The method of claim 10, further comprising the step of contacting the microfilter coated with the positively charged degradable layer with a solution comprising a degradation agent, wherein the degradation agent releases the captured biological material.

12. The method of claim 11, wherein the degradation agent is alginate lyase, ethylenediaminetetraacetic acid (EDTA), egtazic acid (EGTA), sodium citrate, nitrilotriacetic acid, on-hydroxyethylethylenediaminetriacetic acid (hEDTA), oxalic acid, malic acid, rubeanic acid, citric acid or combinations thereof.

13. The method of claim 10, wherein the biological material is a biological material which is negatively charged.

14. The method of claim 13, wherein the biological material which is negatively charged is a bacteria, fungus or virus.

15. A method of preparing a microfiltration device comprising the step of coating a positively charged degradable layer onto a surface of a microfilter.

16. The method of claim 15, further comprising the step of preparing a mixture comprising a crosslinking agent and a polymer substrate solution prior to the coating step.

17. The method of claim 16, wherein the crosslinking agent is a metal salt.

18. The method of claim 17, wherein the metal of the metal salt is selected from the group consisting of calcium (Ca), strontium (Sr), barium (Ba), iron (Fe), copper (Cu), zinc (Zn) potassium (K), sodium (Na) and lithium (Li).

19. The method of claim 10, wherein the coating step comprises the step of spin-coating the mixture onto the surface of the microfilter.

20. A kit comprising the microfiltration device of claim 1, a sampling tube, a drainage tube and a housing container.

21. The method of claim 14, wherein the coating step comprises the step of spin-coating the mixture onto the surface of the microfilter.
